(19) United States
(12) Patent Application Publication
(10) Pub. No.: US 2009/0064768 A1
(43) Pub. Date: Mar. 12, 2009

(54) Devices for Determining the Release Profile of Macromolecules and Methods of Using the Same

(76) Inventors: Emad ALKHAWAM, New City, NY (US); Mankit Ho, West Nyack, NY (US); Jin Qiang Xia, Ridgefield, NJ (US); Satheesh Papineni, Harriman, NY (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/047,051

(22) Filed: Mar. 12, 2008

Related U.S. Application Data
(63) Continuation-in-part of application No. 11/685,520, filed on Mar. 13, 2007.

(51) Int. Cl. G01N 33/15

(52) U.S. Cl. 73/64.56

(57) ABSTRACT

The present invention is directed to devices useful for determining the release profile of a macromolecule from a pharmaceutical composition comprising the macromolecule and methods of using the device. Transport of a macromolecule across a semi-permeable barrier is measured as a function of time using the device, the results of which can correlate with an in vivo release of the macromolecule from a pharmaceutical composition, a bioavailability of the macromolecule, or the stability or purity of the pharmaceutical composition comprising the macromolecule.

### CROSS REFERENCE TO RELATED APPLICATIONS

This application is a continuation-in-part of U.S. application Ser. No. 11/685,520, filed on Mar. 13, 2007, which is hereby incorporated by reference in its entirety.

### BACKGROUND OF THE INVENTION

1. Field of the Invention

Disclosed herein are devices for determining the release profile of macromolecules and methods of using the same.

2. Background

Many macromolecular pharmaceutical dosage forms comprise a suspension of a solid and/or crystalline macromolecule in a pharmaceutically acceptable fluid, gelatin or solid matrix. Macromolecules (i.e., molecules having a molecular weight of about 1,000 Daltons or greater) are notable for their ability to frequently undergo changes in crystalline structure, salvation, hydration, and the like, which can dramatically affect the solubility of a macromolecule, as well as subsequent passage of a macromolecule across various barriers of the body (e.g., skin, cellular membranes, tissue, and the like). Additionally, degradation of a macromolecule can occur during storage due to, for example, interaction with light, a chemical oxidant, and the like that may not induce changes in the tertiary structure of a macromolecule (that can lead to a change in solubility) but may nonetheless render the macromolecule inactive as a therapeutic agent.

Most apparatuses suitable for measuring a dissolution rate of a macromolecule using, for example, paddles, filters, mesh screens, membranes, and the like do not accurately simulate in vivo conditions and are not sufficiently sensitive to detect low concentrations of macromolecules. Moreover, most apparatuses are not capable of isolating physical changes in a macromolecular pharmaceutical composition that can be separately related to the solubility of the macromolecule (i.e., structural stability of the macromolecule) or the bioavailability of the macromolecule (i.e., possible degradation of therapeutic activity).

What is needed is a device and method for effectively and easily determining the stability and activity of a pharmaceutical composition comprising a macromolecule in a straightforward and reproducible manner.

### BRIEF SUMMARY OF THE INVENTION

The present invention is directed to devices and methods of using the devices for determining a release profile for a macromolecule. The present invention is useful for formulating pharmaceutical compositions comprising macromolecules, determining the stability of pharmaceutical formulations comprising macromolecules, testing the bioequivalence of pharmaceutical formulations comprising macromolecules, and combinations thereof.

The present invention is directed to a device comprising:
a first chamber configured to receive a pharmaceutical composition comprising an undissolved macromolecule and a fluid suitable for dissolving the macromolecule to provide a free macromolecule;
a second chamber adjacent to the first chamber, wherein the first chamber and the second chamber are in fluid communication;
a semi-permeable barrier separating the first and the second chambers, wherein a rate of transport of the free macromolecule from the first chamber to the second chamber is determined by an interaction between the free macromolecule and the semi-permeable barrier; and wherein the semi-permeable barrier is adapted to prevent the undissolved macromolecule from passing from the first chamber to the second chamber; and
a sampling port configured to sample the contents of the second chamber.

In some embodiments, the device further comprises a control element configured to apply an external force to the semi-permeable barrier.

In some embodiments, the control element is configured to apply an external force to the semi-permeable barrier chosen from: an electrical potential, a direct electric current, an alternating electric current, a magnetic field, a pressure, a thermal energy, a radiation, a chemical reagent, and combinations thereof.

In some embodiments, the external force is applied to the semi-permeable barrier via the fluid.

In some embodiments, the semi-permeable barrier comprises a material chosen from: a polymer, a glass, a metal, a natural tissue, a synthetic tissue, a biological sample, a composite thereof, and combinations thereof.

In some embodiments, the device further comprises a temperature control element surrounding at least the first chamber.

In some embodiments, the device further comprises a stirrer configured to agitate a fluid within the first chamber.

In some embodiments, the first chamber is located below the second chamber.

In some embodiments, the device further comprises a means for measuring an in situ concentration of a macromolecule present in the second chamber.

In some embodiments, the volume of the first chamber is about 50 mL or less and wherein the volume of the second chamber is about 50 mL or less.

The present invention is also directed to a method of determining a release profile of an undissolved macromolecule from a pharmaceutical composition, the method comprising:
placing a pharmaceutical composition comprising an undissolved macromolecule in a device comprising a first chamber, wherein the first chamber is located adjacent to and in fluid communication with a second chamber, and wherein the first chamber is separated from the second chamber by a semi-permeable barrier adapted to prevent the undissolved macromolecule from passing from the first chamber to the second chamber;
filling the first chamber and the second chamber with a fluid, wherein the fluid interacts with the pharmaceutical composition to provide a free macromolecule in the first chamber, wherein a rate of transport of the free macromolecule from the first chamber to the second chamber is controlled by an interaction between the free macromolecule and the semi-permeable barrier;
measuring the concentration of the free macromolecule in the second chamber as a function of time; and
determining a release profile for the undissolved macromolecule, wherein the release profile is a function of at least the rate at which the free macromolecule is provided in the first chamber and the interaction between the free macromolecule and the semi-permeable barrier.

The present invention is also directed to a method of determining a release profile of a crystalline insulin from a pharmaceutical composition, the method comprising:
placing the pharmaceutical composition in a device comprising a first chamber, wherein the first chamber is located adjacent to and in fluid communication with a second chamber, wherein the first chamber is separated from the second chamber by a semi-permeable barrier adapted to prevent the crystalline insulin from passing from the first chamber to the second chamber;
filling the first chamber and the second chamber with a fluid, wherein the fluid interacts with the pharmaceutical composition to provide a free insulin in the first chamber, wherein a rate of transport of the free insulin from the first chamber to the second chamber is controlled by an interaction between the free insulin and the semi-permeable barrier;
measuring the concentration of the free insulin in the second chamber as a function of time; and
determining a release profile for the crystalline insulin from the pharmaceutical composition, wherein the release profile is a function of at least rate at which the free insulin is provided in the first chamber and the interaction between the free insulin with the semi-permeable barrier.

The present invention is also directed to a method for quantifying the stability and/or quality of a pharmaceutical composition comprising a macromolecule, the method comprising:
providing a first sample of a pharmaceutical composition comprising an undissolved macromolecule;
measuring a release profile for the first sample of the pharmaceutical composition comprising the macromolecule, the measuring comprising:
   (i) placing the pharmaceutical composition comprising an undissolved macromolecule in a device comprising a first chamber, wherein the first chamber is located adjacent to and in fluid communication with a second chamber, and wherein the first chamber is separated from the second chamber by a semi-permeable barrier adapted to prevent the undissolved macromolecule from passing from the first chamber to the second chamber;
   (ii) filling the first chamber and the second chamber with a fluid, wherein the fluid interacts with the pharmaceutical composition to provide a free macromolecule in the first chamber, and wherein a rate of transport of the free macromolecule from the first chamber to the second chamber is controlled by an interaction between the free macromolecule and the semi-permeable barrier;
   (iii)measuring the concentration of the free macromolecule in the second chamber as a function of time; and
   (iv)determining a release profile for the undissolved macromolecule, wherein the release profile is a function of at least the rate at which the free macromolecule is provided in the first chamber and the interaction between the free macromolecule and the semi-permeable barrier;
providing a second sample of the pharmaceutical composition comprising a macromolecule;
measuring a release profile for the second sample of the pharmaceutical composition comprising the macromolecule, the measuring comprising performing (i)-(iv) using the second sample of the pharmaceutical composition; and
comparing the release profile of the second sample with the release profile of the first sample of the pharmaceutical composition to quantify the stability and/or quality of the second sample of the pharmaceutical composition compared to the first sample of the pharmaceutical composition.

In some embodiments, the method further comprises applying an external force to the semi-permeable barrier, wherein the external force affects the interaction between the free macromolecule and the semi-permeable barrier.

In some embodiments, the method further comprises controlling the temperature of at least one of: the fluid, the first chamber, the second chamber, and the semi-permeable barrier.

In some embodiments, the method further comprises emptying the first and second chambers of the fluid; cleaning the first and second chambers and the semi-permeable barrier; and repeating the placing, the filling, the measuring, and the determining.

In some embodiments, the method further comprises emptying the first and second chambers of the fluid; cleaning the first and second chambers; replacing the semi-permeable barrier with a second semi-permeable barrier, wherein the second semi-permeable barrier is different from the semi-permeable barrier; and repeating the placing, the filling, the measuring, and the determining.

In some embodiments, the method further comprises correlating the release profile of the macromolecule with a property of the pharmaceutical composition.

In some embodiments, the method further comprises correlating the release profile of the macromolecule with an in vivo dissolution rate of the pharmaceutical composition.

In some embodiments, the method further comprises correlating the release profile of the macromolecule with a bioavailability of the macromolecule.

In some embodiments, the method further comprises waiting a predetermined time interval between the measuring a release profile for the first sample of the pharmaceutical composition and the measuring a release profile for the second sample of the pharmaceutical composition.

In some embodiments, the first sample of the pharmaceutical composition and the second sample of the pharmaceutical composition are from the same manufacturing lot.

In some embodiments, the device further comprises a temperature control element surrounding at least the first chamber.

In some embodiments, the method further comprises controlling the temperature of at least one of: the fluid, the first chamber, the second chamber, the semi-permeable barrier, and combinations thereof.

Further embodiments, features, and advantages of the present inventions, as well as the structure and operation of the various embodiments of the present invention, are described in detail below with reference to the accompanying drawings.

### BRIEF DESCRIPTION OF THE DRAWINGS/FIGURES

The accompanying drawings, which are incorporated herein and form a part of the specification, illustrate one or more embodiments of the present invention and, together with the description, further serve to explain the principles of the invention and to enable a person skilled in the pertinent art to make and use the invention.

FIGS. 1-5 provide schematic representations of various equilibrium reactions that can occur in various embodiments of the present invention.

FIG. 6 is a cross-sectional schematic representation of a device of the present invention.

FIG. 7 is a graphical representation of the release profiles of several pharmaceutical compositions comprising a macromolecule, as determined by a method of the present invention using a device of the present invention.

FIG. 8 is a graphical representation of the release profiles of several pharmaceutical compositions comprising a macromolecule, as determined by the paddle method.

FIG. 9 is a graphical representation of the release profiles of several pharmaceutical compositions comprising a macromolecule, as determined using a continuous flow-through cell.

FIG. 10 is a graphical representation of the release profiles of a pharmaceutical composition comprising a macromolecule, as determined using a method of the present invention using a device of the present invention, at several different buffer concentrations.

FIG. 11 is a graphical representation of the release profiles of two samples of a pharmaceutical composition comprising a macromolecule, as determined using a method of the present invention, at an initial time point and after storage for 3 months.

One or more embodiments of the present invention will now be described with reference to the accompanying drawings. In the drawings, like reference numbers can indicate identical or functionally similar elements. Additionally, the left-most digit(s) of a reference number can identify the drawing in which the reference number first appears.

### DETAILED DESCRIPTION OF THE INVENTION

Pharmaceutical formulations are frequently formulated to deliver a specific dosage of an active agent to a subject in need thereof, wherein the active agent is released over a predetermined length of time (i.e., at a desired release rate). The release rate of an active agent from a pharmaceutical dosage form over time may be represented in the form of a release profile. Depending on a variety of factors such as, but not limited to, the amount of the active agent to be delivered, the solubility of the active agent, and the desired duration of therapy, pharmaceutical formulations can be designed to reliably deliver a specific active agent with a predetermined release rate. This is generally the case for active agents having a molecular weight less than about 1,000 Daltons. However, difficulties in formulating can arise when an active agent is unstable or can exist in several different crystalline forms, which is the case for many macromolecular active agents. Disclosed herein are devices and methods useful for measuring the release profile of macromolecular active agents from a pharmaceutical dosage form.

As used herein, a “macromolecule” refers to a pharmaceutically active agent having a molecular weight of about 1,000 Daltons or greater. In some embodiments, a macromolecule is a “biological biomacromolecule” or “biomacromolecule,” which as used herein refer to a molecule with a molecular weight of about 1,000 Daltons or greater that can be isolated from an organism or a cellular culture (e.g., a eukaryotic cell culture or a prokaryotic cell culture), or alternatively refer to a biopolymer such as a nucleic acid (e.g., single stranded DNA, double-stranded DNA, single-stranded RNA, and double-stranded RNA), a polypeptide (e.g., a protein), a carbohydrate, a lipid, and combinations thereof.

In some embodiments, a macromolecule comprises a protein. As used herein, “protein” encompasses a singular protein (e.g., a single polypeptide) as well as plural proteins (e.g., dimers, trimers, tetramers, and other multimeric polypeptides). Thus, as used herein, terms including, but not limited to “peptide,” “polypeptide,” “amino acid chain,” or any other term used to refer to a chain or chains of amino acids, are included in the definition of a “protein,” and the term “protein” may be used instead of, or interchangeably with, any of these terms. The term “protein” further includes proteins that have undergone post-translational modifications, for example, glycosylation, acetylation, phosphorylation, amidation, pegylation, or any other derivatization by known protecting/blocking groups, proteolytic cleavage, or modification by non-naturally occurring amino acids. The term “protein” further includes polypeptides that form multimers (e.g., dimers, trimers, tetramers, and the like). The term “protein” further includes fusions proteins (e.g., proteins produced via a gene fusion process in which a protein or fragment thereof is attached to an antibody or antibody fragment). Exemplary fusion proteins for use with the present invention include, but are not limited to, disulfide-linked bifunctional proteins comprised of linked Fc regions from human IgG1 and human IgE; and lymphotoxin beta receptor immunoglobulin GI.

In some embodiments, a macromolecule comprises an antibody. The term “antibody” refers to polyclonal, monoclonal, multispecific, human, humanized or chimeric antibodies, single chain antibodies, humanized antibodies, Fab fragments, F(ab′)2 fragments, fragments produced by a Fab expression library, anti-idiotypic (anti-Id) antibodies, and epitope-binding fragments of any of the above. Antibodies for use with the present invention can be from any animal origin including birds and mammals (e.g., human or non-human, such as murine, donkey, ship rabbit, goat, guinea pig, camel, horse, chicken and the like). As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins. See, e.g., U.S. Pat. No. 5,939,598.

In some embodiments, the term “antibody” refers to a monoclonal antibody. The term “antibody” also refers to immunoglobulin molecules and immunologically active portions of immunoglobulin molecules, i.e., macromolecules that contain an antigen binding site that immunospecifically binds an antigen. Exemplary antibodies for use with the present invention include, but are not limited to, natalizmab, humanized Anti-Alpha V Beta-6 monoclonal antibody, IgG1, IgG2, IgG3, IgG4, humanized anti-VLA1 IgG1 kappa monoclonal antibody, huB3F6, and combinations thereof.

In some embodiments, a macromolecule comprises a nanoparticle (e.g., a metallic nanoparticle, a metal-organic nanoparticle, and combinations thereof having a mean diameter of about 10 nm to about 500 nm). For example, a macromolecule can be adsorbed to a nanoparticle, or form a complex with a nanoparticle.

Specific macromolecules suitable for use with the present invention include, but are not limited to, human growth hormone, insulin, insulin-like growth factor, oxytocin, epidermal growth factor, ferritin, hemoglobin, myoglobin, fibrin, thrombin, factor XIII, factor VIII, von Willebrand factor, protein C, protein Z, protein Z-related protease inhibitor, protein S, C1-inhibitor, C3-convertase, serum albumin, serum amyloid P component, follicle-stimulating hormone, erythropoietin, granulocyte colony-stimulating factor (e.g., filgrastim and pegfilgrastim), α-glactosidase A, α-L-iduronidase (e.g., rhIDU and laronidase), N-acetylgalactosamine-4-sulfatase (e.g., rhASB and galsulfase, DNAse, tissue plasminogen activator, glucocerebrosidase, interferon (e.g., interferon-β-1a and interferon β-lb), bovine somatotropin, porcine somatotropin, bovine chymosin, bevacizumab, bortezomib, erlotinib, pegaptanib, ranibizumab, sorafenib, lenalidomide, sunitinib, envelope protein of the hepatitis B virus, and combinations thereof.

In some embodiments, a macromolecule comprises insulin. Insulin suitable for use with the present invention include, but are not limited to, insulin N, insulin R, insulin analogs (e.g., insulin glargine, insulin detemir, etc.), and mixtures thereof.

As used herein, an “undissolved macromolecule” refers to a macromolecule that is not dissolved or otherwise in solution. In some embodiments, an undissolved macromolecule can refer to a crystalline, a polycrystalline, a co-crystalline, an amorphous, or a granular form of a macromolecule, and combinations thereof. An undissolved macromolecule can be in a micronized form. In some embodiments, an undissolved macromolecule is blended with pharmaceutically acceptable excipients in a liquid, solid, or gelled dosage form such as, but not limited to, a suspension, a gel, a capsule, a paste, a cream, a tablet, a troche, and the like, and any other forms known to a person of ordinary skill in the art. In some embodiments, an undissolved macromolecule refers to an isotonic suspension comprising a crystalline and/or micronized macromolecule.

As used herein, a “free macromolecule” refers generally to a dissolved macromolecule or a macromolecule that is “in solution” or otherwise solubilized, and is fluid (i.e., capable of flowing). In some embodiments, a free macromolecule includes dimers, trimers, tetramers, and other multimeric species capable of being formed by macromolecules. Additionally, the term “free macromolecule” also refers to solubilized or dissolved complexes formed between a macromolecule and another species, moiety, and the like.

### Devices

Disclosed herein is a device suitable for measuring a release profile of a macromolecule from a pharmaceutical composition comprising a macromolecule. Pharmaceutical compositions suitable for measurement by the present invention include, but are not limited to, suspensions, solutions, creams, gels, amorphous solids, crystalline solids, crystalline gels, crystalline suspensions, and combinations thereof.

The present invention is directed to a device comprising:
a first chamber configured to receive a pharmaceutical composition comprising an undissolved macromolecule and a fluid suitable for dissolving the macromolecule to provide a free macromolecule;
a second chamber adjacent to the first chamber, wherein the first chamber and the second chamber are in fluid communication;
a semi-permeable barrier separating the first and the second chambers, wherein a rate of transport of the free macromolecule from the first chamber to the second chamber is determined by an interaction between the free macromolecule and the semi-permeable barrier; and wherein the semi-permeable barrier is adapted to prevent the undissolved macromolecule from passing from the first chamber to the second chamber; and
a sampling port configured to sample the contents of the second chamber.

The first chamber is located proximate to or adjacent to the second chamber. In some embodiments, the first chamber is located below the second chamber. In some embodiments, the first chamber and second chamber are connected by a channel, tube, pipe, capillary, and the like, wherein the connecting element does not add significantly to the enclosed volume of the device (e.g., a connecting element is about 50% or less, about 40% or less, about 30% or less, about 20% or less, about 10% or less, or about 5% or less of the overall volume of the device).

The first chamber and the second chamber are in fluid communication. As used herein, “fluid communication” refers to the ability for a fluid added to either of the first chamber or the second chamber to flow between the chambers. The devices of the present invention have a semi-permeable barrier separating the first and second chambers. Therefore, fluid communication between the first and second chambers can be achieved by fluid flowing through the semi-permeable barrier from the first chamber to the second chamber, or vice versa. Fluid communication can also refer to the ability of an analyte, a free macromolecule, an ion, or some other moiety, either dissolved or suspended in the fluid to pass between the first and second chambers through the semi-permeable barrier with or without passage of the fluid between the chambers. Thus, in some embodiments the permeability of semi-permeable barrier can determine the degree of fluid communication between the first and second chambers.

As used herein, a “fluid” refers to a continuous amorphous composition that tends to flow and to conform to the outline of a container. Fluids for use with the present invention include, but are not limited to: materials capable of forming solutions, suspensions, mixtures, and the like with a macromolecule. Fluids for use with the present invention include, but are not limited to, water, alcohols (e.g., ethanol, butanol, glycols, and the like), ketones (e.g., acetone, methylethylketone, and the like), esters (e.g., ethylacetate, and the like), halogenated solvents (methylene chloride, 1,2-dichloroethane, and the like), ethers (e.g., diethylether, propylene glycol dimethyl ether, and the like), liquid crystalline substances, ionic liquids, amides (e.g., dimethylformamide, N-methylpyrrolidone, and the like), and combinations thereof, and other fluids known to persons of ordinary skill in the art. In some embodiments, a fluid comprises a pharmaceutically acceptable fluid. Fluids for use with the present invention also include biological fluids and synthetic variants thereof, such as, but not limited to: saliva, blood, gastric juices, intestinal fluids, mucous, interstitial fluid, and the like.

The fluid for use with the present invention can also include excipients chosen from: surfactants, solubilizers, anti-oxidants, polymers, ionic excipients, salts, acids, bases, and the like, and combinations thereof, capable of substantially dissolving in the fluid medium. In some embodiments, the fluid medium can contain about 100% or less, about 50% or less, about 40% or less, about 25% or less, about 10% or less, or about 5% or less by weight of an excipient that is not substantially soluble in the fluid medium, so long as the macromolecule is capable of dissolving in the fluid medium in the presence of the substantially insoluble excipient.

In some embodiments, the fluid medium has a pH of about 5 to about 9. In some embodiments, the fluid medium has a minimum pH of about 5 to about 8.5, about 5 to about 8, about 5 to about 7.5, about 5 to about 7, about 5 to about 6.5, about 5.5 to about 9, about 5.5 to about 8.5, about 5.5 to about 8, about 5.5 to about 7.5, about 5.5 to about 7, about 6 to about 9, about 6 to about 8.5, about 6 to about 8, about 6 to about 7.5, about 6 to about 7, about 6.5 to about 9, about 6.5 to about 8.5, about 6.5 to about 8, about 6.5 to about 7.5, about 6.5 to about 7, about 7 to about 9, about 7 to about 8.5, about 7 to about 8, about 7.5 to about 9, about 7.5 to about 8.5, or about 8 to about 9.

Buffers suitable for use with the present invention include any pharmaceutically acceptable buffer compositions such as, but not limited to, a phosphate buffer (e.g., monobasic sodium phosphate, dibasic sodium phosphate, tribasic sodium phosphate, monobasic potassium phosphate, dibasic potassium phosphate, tribasic potassium phosphate, monobasic ammonium phosphate, dibasic ammonium phosphate, tribasic ammonium phosphate, dibasic calcium phosphate, and phosphate buffered saline), a citrate buffer, a borate buffer, a phthalate buffer, an acetate buffer, a (hydroxymethyl)aminoethane buffer (e.g., TRIZMA , Sigma Chemical Co., St. Louis, Mo.), and combinations thereof

Not being bound by any particular theory, ions present in a buffered solution can interact with ions present in an undissolved macromolecule to stabilize the macromolecule, thereby increasing the dissolution rate of the macromolecule from a pharmaceutical composition.

Devices of the present invention comprise a semi-permeable barrier. As used herein, “semi-permeable” refers to a barrier that selectively allows certain fluids and certain compounds, molecules, elements, ions, polymers, and the like to pass across the barrier while excluding others, wherein the permeability of the barrier to a specific compound, molecule, element, ion, polymer, and the like is attributable to one or more properties of the compound, molecule, element, ion, polymer, and the like and an interaction between the compound, molecule, element, ion, polymer, and the like with the surface, three-dimensional shape, or some other property of the semi-permeable barrier.

The semi-permeable barrier is adapted to prevent an undissolved macromolecule present in the first chamber from passing from the first chamber to the second chamber.

An undissolved macromolecule can be prevented from passing through the semi-permeable barrier due to size, shape, electrostatic repulsion, magnetic repulsion, chemisorption, physisorption, and the like, and other interactions between macromolecules and surfaces known to persons of ordinary skill in the art.

In some embodiments, the semi-permeable barrier comprises a material chosen from: a polymer (e.g., a polyester, a polyether, a cellulose, a polyethersulfone, a nylon, a polyacrylate, a polyalkylacrylate, a polyalkylene, a polyimide, a polycarbonate, a polyphenylene, a polynaphthalene, a polysilsesquioxance, a polysiloxane, a polysaccharide, a polypeptide, derivatives thereof, porous variants thereof, and substituted variants thereof (e.g., halogenated and alkylated variants thereof, and the like), ionomers thereof, and copolymers thereof), a glass (e.g., a silicate, a borosilicate, an aluminate, a zeolite, and porous variants thereof), a metal (e.g., a transition metal, a group 13 metal, a group 14 metal, a group 15 metal, a group 16 metal, and alloys thereof), a natural tissue (e.g., a vertebrate tissue or an invertebrate tissue), a synthetic tissue (e.g., a hydrogel tissue, a nanotube tissue, a polymeric tissue, and the like, and combinations thereof), a biological sample (e.g., a plant sample, an animal tissue sample cultivated in vitro, and the like), and combinations thereof. In some embodiments, the semi-permeable barrier is a polymer chosen from cellulose acetate, polyethersulfone, a polycarbonate, and combinations thereof.

In some embodiments, a semi-permeable barrier can comprise a material (e.g., a polymer, a membrane, a mesh screen, a filter, and the like) capable of maintaining a static electrical charge, thereby creating a potential gradient within the first and second chambers. In some embodiments a static electrical charge on the semi-permeable barrier repels like-charged species, macromolecules, and complexes thereof, attracts oppositely-charged species, macromolecules, and complexes thereof, and permits charge-neutral species, macromolecules, and complexes thereof to pass through the semi-permeable barrier.

In some embodiments, a semi-permeable barrier comprises a material having a chemical functional group capable of interacting with a macromolecule, a functional group thereof, a pendant group thereof, and the like. In some embodiments, a semi-permeable barrier is derivatized with a chemical functional group chosen from: hydroxyl, alkoxyl, thiol, alkylthio, silyl, alkylsilyl, alkylsilenyl, siloxyl, primary amino, secondary amino, tertiary amino, carbonyl, alkylcarbonyl, aminocarbonyl, carbonylamino, carboxy, and combinations thereof.

As used herein, “alkyl,” by itself or as part of another group, refers to straight and branched chain hydrocarbons of up to 60 carbon atoms, such as, but not limited to, octyl, decyl, dodecyl, hexadecyl, and octadecyl.

As used herein, “alkenyl,” by itself or as part of another group, refers to a straight and branched chain hydrocarbons of up to 60 carbon atoms, wherein there is one, two, three, or more double bonds between two of the carbon atoms in the chain, and wherein the double bond can be in either of the cis or trans configurations, including, but not limited to, 2-octenyl, 1-dodecenyl, 1-8-hexadecenyl, 8-hexadecenyl, and 1-octadecenyl.

As used herein, “alkynyl,” by itself or as part of another group, refers to straight and branched chain hydrocarbons of up to 60 carbon atoms, wherein there is one, two, three, or more triple bonds between two of the carbon atoms in the chain, including, but not limited to, 1-octynyl and 2-dodecynyl.

As used herein, “aryl,” by itself or as part of another group, refers to cyclic, fused cyclic, and multi-cyclic aromatic hydrocarbons containing up to 60 carbons in the cyclic portion, such as, but not limited to, phenyl, naphthyl, anthracenyl, fluorenyl, tetracenyl, pentacenyl, hexacenyl, perylenyl, terylenyl, quaterylenyl, coronenyl, and fullerenyl.

As used herein, “aralkyl” or “arylalkyl,” by itself or as part of another group, refers to alkyl groups as defined above having one, two, three, or more aryl substituents, such as, but not limited to, benzyl, phenylethyl, and 2-naphthylmethyl. Similarly, the term “alkylaryl,” as used herein by itself or as part of another group, refers to an aryl group, as defined above, having an alkyl substituent, as defined above.

As used herein, “heteroaryl,” by itself or as part of another group refers to cyclic, fused cyclic and multicyclic aromatic groups containing up to 60 atoms in the ring portions, wherein the atoms in the ring(s), in addition to carbon, include one, two, three, or more heteroatoms. The term “heteroatom” is used herein to mean an oxygen atom (“O”), a sulfur atom (“S”) or a nitrogen atom (“N”). Additionally, the term heteroaryl also includes N-oxides of heteroaryl species that containing a nitrogen atom in the ring. Examples of heteroaryl species include, but are not limited to, pyrrolyl, pyridyl, pyridyl N-oxide, thiophenyl, and furanyl.

Any one of the above groups can be further substituted with one, two, three, or more of the following substituents: hydroxyl, alkoxyl, thiol, alkylthio, silyl, alkylsilyl, alkylsilenyl, siloxyl, primary amino, secondary amino, tertiary amino, carbonyl, alkylcarbonyl, aminocarbonyl, carbonylamino, carboxy, halo, perhalo, alkylenedioxy, and combinations thereof.

As used herein, “hydroxyl,” by itself or as part of another group, refers to an (—OH) moiety.

As used herein, “alkoxyl,” by itself or as part of another group, refers to one or more alkoxyl (—OR) moieties, wherein R is chosen from the alkyl, alkenyl, alkynyl, aryl, aralkyl, and heteroaryl groups described above.

As used herein, “thiol,” by itself or as part of another group, refers to an (—SH) moiety.

As used herein, “alkylthio,” refers to an (—SR) moieties, wherein R is chosen from the alkyl, alkenyl, alkynyl, aryl, aralkyl, and heteroaryl groups described above.

As used herein, “silyl,” by itself or as part of another group, refers to an (—SiH₃) moiety.

As used herein, “alkylsilyl,” by itself or as part of another group, refers to an (—Si(R)ₓH_{y}) moiety, wherein 1≦x≦3 and y=3−x, and wherein R is independently chosen from the alkyl, alkenyl, alkynyl, aryl, aralkyl, and heteroaryl groups described above.

As used herein, “alkylsilenyl,” by itself or as part of another group, refers to a (—Si(═R)H) moiety, wherein R is chosen from the alkyl, alkenyl, alkynyl, aryl, aralkyl, and heteroaryl groups described above.

As used herein, “siloxyl,” by itself or as part of another group, refers to a (—Si(OR)ₓR¹_{y}) moiety, wherein 1≦x≦3 and y=3−x, wherein R and R¹ are independently chosen from hydrogen and the alkyl, alkenyl, alkynyl, aryl, aralkyl, and heteroaryl groups described above.

As used herein, “primary amino,” by itself or as part of another group, refers to an (—NH₂) moiety.

As used herein, “secondary amino,” by itself or as part of another group, refers to an (—NRH) moiety, wherein R is chosen from the alkyl, alkenyl, alkynyl, aryl, aralkyl, and heteroaryl groups described above.

As used herein, “tertiary amino,” by itself or as part of another group, refers to an (—NRR¹) moiety, wherein R and R¹ are independently chosen from the alkyl, alkenyl, alkynyl, aryl, aralkyl, and heteroaryl groups described above.

As used herein, “carbonyl,” by itself or as part of another group, refers to a (C═O) moiety.

As used herein, “alkylcarbonyl,” by itself or as part of another group, refers to a (—C(═O)R) moiety, wherein R is independently chosen from hydrogen and the alkyl, alkenyl, alkynyl, aryl, aralkyl, and heteroaryl groups described above.

As used herein, “aminocarbonyl,” by itself or as part of another group, refers to a (—C(═O)NRR¹) moiety, wherein R and R¹ are independently chosen from hydrogen and the alkyl, alkenyl, alkynyl, aryl, aralkyl, and heteroaryl groups described above.

As used herein, “carbonylamino,” by itself or as part of another group, refers to a (—N(R)C(═O)R¹) moiety, wherein R and R¹ are independently chosen from hydrogen and the alkyl, alkenyl, alkynyl, aryl, aralkyl, and heteroaryl groups described above.

As used herein, “carboxy,” by itself or as part of another group, refers to a (—COOR) moiety, wherein R is independently chosen from hydrogen and the alkyl, alkenyl, alkynyl, aryl, aralkyl, and heteroaryl groups described above.

A chemical functional group present on a semi-permeable barrier for use with the present invention can impart a “surface characteristic” to the semi-permeable barrier. In some embodiments, the semi-permeable barrier is selected based on its surface energy. Surface free energy is generally the work required to increase the area of a material by one unit area, and also relates to the wettability of a material. Surface energy can be determined using, for example, a contact angle goniometer and the like, or by other methods known to persons of ordinary skill in the art. In some embodiments, a semi-permeable barrier for use with the present invention has a surface energy of about 15 dynes/cm to about 50 dynes/cm.

Generally, the chemical functionality of the semi-permeable barrier is hydrophilic or hydrophobic. As used herein, a hydrophilic semi-permeable barrier is that which water forms a contact angle, Θ, wherein Θ≦90°; and a hydrophobic semi-permeable barrier is that on which water forms a contact angle, Θ, wherein Θ>90°.

A hydrophilic semi-permeable barrier can comprise: a hydrogen-bond donating functional group, a hydrogen-bond receiving functional group, a chemically reactive functional group, and combinations thereof As used herein, a hydrogen-bond donating semi-permeable barrier has an exposed functional group such as, but not limited to, —NH₂, —N(R)H, —OH, and the like, wherein R is defined above, or a metal hydride group capable of forming a hydrogen bond. As used herein, a hydrogen-bond receiving semi-permeable barrier has a functional group that includes an exposed N, O, or F atom having a lone pair of electrons, or a metal group capable of forming a hydrogen bond with water. As used herein, a “chemically reactive semi-permeable barrier” has an exposed functional group other than an alkyl, fluoroalkyl or perfluoroalkyl group.

Functional groups suitable for imparting hydrophobicity to a semi-permeable barrier include: but are not limited to, halo, perhalo, alkyl, alkenyl, alkynyl, aryl, arylalkyl, heteroaryl, and alkylsilyl groups (as defined above), and combinations thereof. Substituted alkyl, alkenyl, alkynyl, aryl, arylalkyl, heteroaryl, and alkylsilyl groups (as defined above), can also be suitable for imparting hydrophobicity to a semi-permeable barrier, wherein the functional groups present in the material are not exposed at the surface of the semi-permeable barrier. For example, hydrogen-bond donating and accepting groups, and the like, can be present within the pores or cavities of a semi-permeable barrier having a hydrophobic surface.

As used herein, “halo,” by itself or as part of another group, refers to any of the above alkyl, alkenyl, alkynyl, aryl, aralkyl, and heteroaryl groups wherein one or more hydrogens thereof are substituted by one or more fluorine, chlorine, bromine, or iodine atoms.

As used herein, “perhalo,” by itself or as part of another group, refers to any of the above alkyl, alkenyl, alkynyl, aryl, aralkyl, and heteroaryl groups wherein all of the hydrogens thereof are substituted by fluorine, chlorine, bromine, or iodine atoms.

Functional groups suitable for imparting hydrophilicity to a semi-permeable barrier include: but are not limited to, hydroxyl, alkoxyl, thiol, silyl, siloxyl, primary amino, secondary amino, carbonyl, alkylcarbonyl, aminocarbonyl, carbonylamino, carboxy, alkylenedioxy, and combinations thereof. Not being bound by any particular theory, alkylsilyl, alkylsilenyl, siloxyl, primary amino, secondary amino, tertiary amino, alkylcarbonyl, aminocarbonyl, carbonylamino, and carboxy functional groups can also impart hydrophobicity to a surface depending on the presence and length of an —R group attached to the functional group. Generally, increasing the length of an alkyl, alkenyl, or alkynyl chain will increase the hydrophobicity of the semi-permeable barrier.

As used herein, “alkylenedioxy,” by itself or as part of another group, refers to a ring and is especially C₁₋₄ alkylenedioxy. Alkylenedioxy groups can optionally be substituted with halogen (especially fluorine). Typical examples include methylenedioxy (—OCH₂O—) or difluoromethylenedioxy (—OCF₂O—).

The semi-permeable barrier permits fluid to flow from the first chamber to the second chamber. In addition, the semi-permeable barrier prevents an undissolved macromolecule from passing from the first chamber through the semi-permeable barrier to the second chamber. In some embodiments, the semi-permeable barrier comprises a continuous or semi-continuous network of pores. In some embodiments, the semi-permeable barrier is a cellulose acetate membrane having a pore size of about 200 nm to about 450 nm.

In some embodiments, the semi-permeable barrier comprises a continuous or semi-continuous network of pores having a mean diameter of about 0.7 nm to about 100 μm. In some embodiments, the semi-permeable barrier comprises a continuous or semi-continuous network of pores having a mean diameter of about 1 nm to about 100 μm, about 1 nm to about 50 μm, about 1 nm to about 25 μm, about 1 nm to about 10 μm, about 1 nm to about 1 μm, about 1 nm to about 500 nm, about 1 nm to about 250 nm, about 1 nm to about 100 nm, about 1 nm to about 50 nm, about 1 nm to about 10 nm, about 10 nm to about 100 μm, about 10 nm to about 50 μm, about 10 nm to about 10 μm, about 10 nm to about 1 μm, about 10 nm to about 500 nm, about 10 nm to about 250 nm, about 10 nm to about 100 nm, about 10 nm to about 50 nm, about 50 nm to about 100 μm, about 50 nm to about 50 μm, about 50 nm to about 10 μm, about 50 nm to about 1 μm, about 50 nm to about 500 nm, about 100 nm to about 100 μm, about 100 nm to about 50 μm, about 100 nm to about 10 μm, about 100 nm, to about 1 μm, about 100 nm to about 500 nm, about 200 nm to about 100 μm, about 200 nm to about 10 μm, about 200 nm, to about 1 μm, about 200 nm to about 500 nm, or about 250 nm, about 450 nm, about 500 nm, or about 1 μm.

Not being bound by any particular theory, the rate of transport of a free macromolecule from the first chamber to the second chamber is controlled by one or more properties of the semi-permeable barrier and/or the magnitude of an external force applied to the semi-permeable barrier.

In some embodiments, the device comprises a control element configured to apply an external force to the semi-permeable barrier. In some embodiments, the control element is configured to apply an external force chosen from: an local electrical potential, a direct electric current, an alternating electric current, a local magnetic potential, a pressure (e.g., a hydrostatic pressure or a fluid dynamic pressure), a thermal energy, a radiation, a chemical reagent, and combinations thereof to the semi-permeable barrier.

An external force can be applied to the semi-permeable barrier in a constant manner, or in a time-varying manner (e.g., the amplitude of the external force varies over time in an increasing, a decreasing, or a periodic manner, or any combination thereof).

In some embodiments, a local electrical potential of about −2 V to about 2 V, about −1.5 V to about 1.5 V, about −1 V to about 1 V, about −0.5 V to about 0.5 V, about −0.1 V to about 0.1 V, about −10 mV to about 10 mV, about −1 mV to about 1 mV, or about −0.1 mV to about 0.1 mV is applied to the semi-permeable barrier.

In some embodiments, a continuous or pulsed direct electric current or an alternating electric current of about 1 pA to about 1 μA, about 1 pA to about 1 nA, or about 1 nA is applied to a semi-permeable barrier.

In some embodiments, the external force is applied to the semi-permeable barrier via the fluid present in either one of the first chamber or second chamber. For example, the external force can comprise, but is not limited to, a hydrostatic pressure inside the first and/or second chambers of about 1 Pa to about 5 Pa, about 1 Pa to about 3 Pa, about 1 Pa to about 2 Pa, about 1 Pa to about 1.5 Pa, or about 1 Pa. Additionally, a fluid can transport a chemical reagent to the semi-permeable barrier such as, for example, a reducing agent, an oxidizing agent, an acid, a base, a halogenation agent, and the like, that can modify the surface of the semi-permeable barrier or react with the semi-permeable barrier.

In some embodiments, the device further comprises a temperature control element surrounding at least the first chamber. Suitable temperature controllers include resistive heating elements, circulating chilling or heating elements, and the like, and any other temperature control elements known to persons of ordinary skill in the art. The temperature of the first and/or second chamber can be controlled at about −25° C. to about 90° C., about −25° C. to about 70° C., about −25° C. to about 25° C., about −10° C. to about 50° C., about −10° C. to about 25° C., about 0° C. to about 90° C., about 0° C. to about 50° C., about 0° C. to about 25° C., about 10° C. to about 90° C., about 10° C. to about 50° C., about 10° C. to about 25° C., about 20° C. to about 90° C., about 20° C. to about 70° C., about 20° C. to about 50° C., about 25° C., about 30° C., about 35° C., or about 37° C. In some embodiments, the temperature of the first and/or second chamber can be controlled to a maximum temperature of about 90° C., about 80° C., about 70° C., about 60° C., about 50° C., about 40° C., or about 37° C.

In some embodiments, the device further comprises a stirrer. In some embodiments, a stirrer is configured to agitate a fluid within the first chamber, a fluid within the second chamber, and combinations thereof Stirrers suitable for use with the present invention include, but are not limited to, a mechanical stirrer (e.g., a paddle, a wire, a rotatable coil, and the like), a magnetic stirrer, a piston, a sonicator, and other mixing and/or stirring elements known to a person of ordinary skill in the mixing arts. In some embodiments, the stirrer comprises a stainless steel rotatable coil sized to match the dimensions of the first and/or second chamber. In some embodiments, a magnetic stirrer is attached to the bottom of the rotatable coil.

In some embodiments, the volume of the first chamber and/or the second chamber is about 50 mL or less. In some embodiments, the first chamber and/or the second chamber has a maximum volume of about 40 mL, about 30 mL, about 25 mL, about 20 mL, about 15 mL, about 10 mL, about 5 mL, about 2 mL, about 1 mL, or about 0.5 mL. In some embodiments, the first chamber and/or the second chamber has a minimum volume of about 0.1 mL, about 0.2 mL, about 0.5 mL, about 1 mL, about 2 mL, about 5 mL, about 10 mL, or about 15 mL. In some embodiments, the volume of the first chamber and/or the second chamber is about 1 mL to about 20 mL, about 1 mL to about 15 mL, about 1 mL to about 10 mL, or about 1 mL to about 5 mL.

The device further comprises a sampling port configured to sample the contents of the second chamber. The sampling port can be of any shape, size and configuration suitable for removing a fluid sample from the second chamber or suitable for inserting a probe, a capillary, a pipette, a syringe, and the like into the second chamber to obtain a sample therefrom.

In some embodiments, the device further comprises a means for measuring an in situ concentration of a macromolecule present in the second chamber. For example, the device can include a means for measuring an electrical potential, a spectrophotometer, a high-performance liquid chromatograph, a gas chromatograph, a gravimetric measuring device, a mass spectrometer, and combinations thereof, and any other quantitative analytical devices and instruments known to persons of ordinary skill in the analytical arts. When the concentration of a macromolecule is not directly measured, then the signal obtained by the means of measuring can be correlated with the macromolecule concentration.

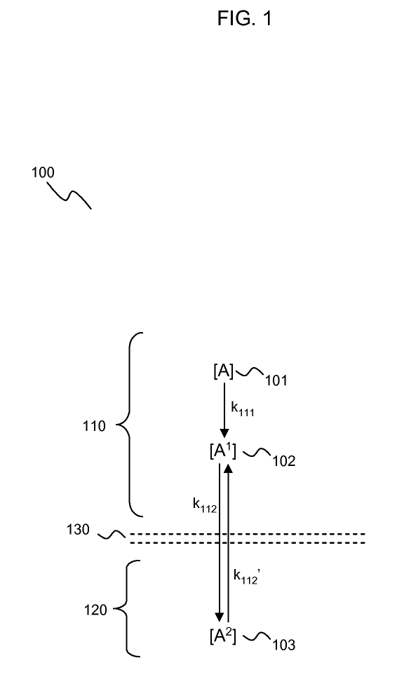

FIGS. 1-5 provide schematic representations, 100, 200, 300, 400 and 500, respectively, of reactions related to embodiments of the present invention and that can occur in a device of the present invention related to the determination of a release profile for a pharmaceutical composition comprising a macromolecule. Referring to FIG. 1, a pharmaceutical composition comprising an undissolved macromolecule, 101, is placed in a first chamber, 110, of a device of the present invention. The concentration of undissolved macromolecule in the first chamber is indicated by [A]. Upon injection of a fluid medium into the first chamber the undissolved macromolecule, 101, undergoes irreversible dissolution in the fluid medium with a rate indicated by k₁₁₁, to form a free macromolecule, 102, having a concentration in the first chamber of [A¹]. The free macromolecule, 102, passes through the semi-permeable barrier, 130, to the second chamber, 120, with a rate indicated by k₁₁₂, to provide a free macromolecule in the second chamber, 103, having a concentration [A²]. The free macromolecule present in the second chamber, 103, can also pass back across the semi-permeable barrier, 130, to the first chamber with a rate indicated by k₁₁₂′. The concentration of the free macromolecule in the second chamber, [A²], is measured as a function of time to determine a release profile of the undissolved macromolecule from the pharmaceutical composition, 101.

Not being bound by any particular theory, when k₁₁₂>>k₁₁₁, the release profile of the undissolved macromolecule from the pharmaceutical composition, 101, is approximately equivalent to the dissolution rate of the undissolved macromolecule in the fluid medium of the first chamber. Conversely, when k₁₁₁>>k₁₁₂, the release profile of an undissolved macromolecule from the pharmaceutical composition, 101, is approximately equivalent to the rate that the free macromolecule, 102, passes through the semi-permeable barrier from the first chamber to the second chamber, k₁₁₂.

In some embodiments, a series of release profiles can be determined wherein at least one of the fluid medium, the semi-permeable barrier is varied. Not being bound by any particular theory, such variability can be used to isolate whether the release profile for a given macromolecule is rate-limited by the release and dissolution of the undissolved macromolecule from a pharmaceutical composition or transport of a free macromolecule across a semi-permeable barrier. For example, a change in the three-dimensional structure of a macromolecule can either increase or decrease the solubility of the macromolecule in a first fluid medium. Therefore, determining release profiles for a macromolecular pharmaceutical composition in several different fluids and/or using several different barriers can provide information relating to, for example, structural changes in the macromolecule (e.g., denaturing), degradation of the macromolecule (e.g., oxidation), and the like that may occur during storage. Thus, in some embodiments the present invention is directed to determining the release profile of a macromolecule from a pharmaceutical composition under varied conditions such as, but not limited to, varied semi-permeable barriers (e.g., having varied pore size, varied functional groups, and the like), varied fluids (e.g,. varied dielectric constant, polarity, functional groups, and the like), a range of pH values, a range of temperatures, and the like, and combinations thereof.

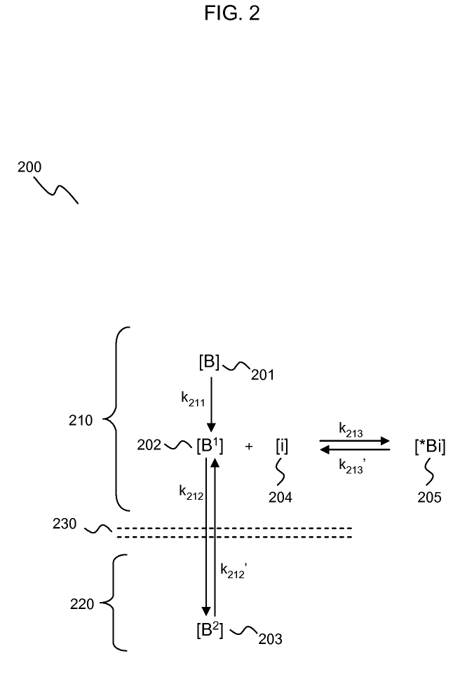

Referring to FIG. 2, a pharmaceutical composition comprising an undissolved macromolecule, 201, is placed in a first chamber, 210, of a device of the present invention. The concentration of undissolved macromolecule in the first chamber is given by [B]. Upon injection of a fluid medium into the first chamber the macromolecule, B, undergoes irreversible dissolution in the fluid medium with a rate indicated by k₂₁₁, to form a free macromolecule in the first chamber, 202, having a concentration [B¹]. A species, 204, having a concentration [i] that is capable of reversibly interacting with the free macromolecule, 202, to induce a structural, conformational, ionic, solubility, or some other change in the free macromolecule is present in the first chamber, 210. Reaction between the free macromolecule, 202, and the species, 204, with a rate given by k₂₁₃ yields the free or undissolved complex, 205, having a concentration in the first chamber of [*Bi]. The structural, conformational, ionic, solubility, or some other change induced by the species, 204, renders the complex, 205, incapable of crossing the semi-permeable barrier, 230. However, in some embodiments, the complex, 205, can undergo a dissociation reaction with a rate, k₂₁₃′, to yield the free macromolecule, 202, and the species, 204. The free macromolecule in the first chamber, 202, passes through the semi-permeable barrier, 230, with a rate indicated by k₂₁₂, to provide a free macromolecule in the second chamber, 203. In some embodiments, the free macromolecule can reversibly cross the semi-permeable barrier, transport from the second chamber back to the first chamber having a rate given by k₂₁₂′. The concentration of the free macromolecule in the second chamber, [B²], can be measured as a function of time to obtain a release profile for the pharmaceutical composition.

Not being bound by any particular theory, the embodiments depicted in FIG. 2 can be useful for determining the stability and/or shelf-life of a pharmaceutical composition comprising a macromolecule that is readily soluble in a fluid medium, and/or for determining the release profile of a macromolecule that in the absence of a complexing species, 204, undergoes rapid passage across a semi-permeable barrier (e.g., the rate k₂₁₂ is very fast). When the complexation reaction between the free macromolecule and the species 204 is irreversible, then such a system can be useful for determining the release profile of a macromolecule that rapidly passes through and back across a semi-permeable barrier (e.g., k₂₁₂≈k₂₁₂′>>k₂₁₁).

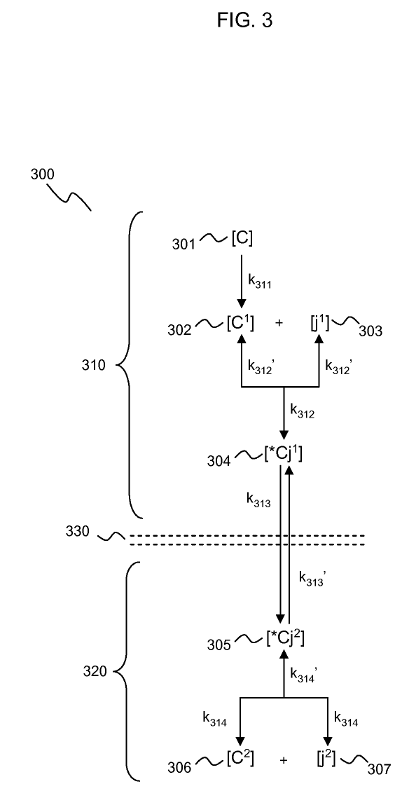

Referring to FIG. 3, a pharmaceutical composition comprising an undissolved macromolecule, 301, is placed in a first chamber, 310, of a device of the present invention. The concentration of undissolved macromolecule in the first chamber is indicated by [C]. Upon injection of a fluid medium into the first chamber the macromolecule undergoes irreversible dissolution in the fluid medium with the rate k₃₁₁, to yield in the first chamber a free macromolecule, 302, having a concentration [C¹]. A semi-permeable barrier, 330, is selected such that passage of the free macromolecule from the first chamber, 310, through the semi-permeable barrier, 330, to the second chamber, 320, does not substantially occur. A species, 303, capable of complexing with the free macromolecule, 302, can be introduced into the first chamber in a concentration given by [j¹], and wherein complexation between the species, 303, and the free macromolecule, 302, occurs with a rate given by k₃₁₂ to yield the complex, 304, having a concentration in the first chamber, [*Cj¹]. The complex, 304, can have a structure, conformation, size, ionic charge, solubility, or some other physical and/or chemical property that is different from the free macromolecule, 302. Complexation between the free macromolecule, 302, and the species, 303, can be reversible (e.g., k₃₁₂≈k₃₁₂′) or irreversible (e.g., k₃₁₂>>k₃₁₂′). The structural, conformational, size, ionic, solubility, or some other change induced by the species, 303, renders the complex, 304, capable of passing through the semi-permeable barrier, 330, with a rate indicated by k₃₁₃, to provide in the second chamber, 320, the complex, 305, with a concentration [*Cj²]. From the second chamber, 320, the complex, 305, can pass back to the first chamber with a rate k₃₁₃′, or dissociate with a rate indicated by k₃₁₄, in the second chamber to yield the free macromolecule, 306, and the species, 307, with concentrations of [C²] and [j²], respectively. It is also possible for the free macromolecule, 306, and the complex, 307, to re-associate to form the complex, 305, in the second chamber, as indicated by the rate, k₃₁₄′. Any one of the concentrations of the free macromolecule, 306, the species, 307, the complex, 305, and combinations thereof, in the second chamber can be measured as a function of time to determine a release profile for a pharmaceutical composition comprising the undissolved macromolecule, 301, in the first chamber.

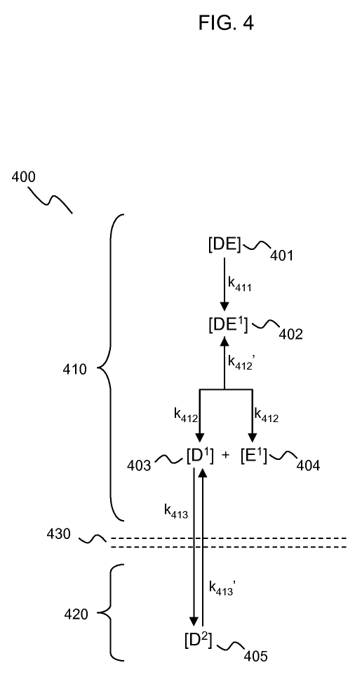

Referring to FIG. 4, a pharmaceutical composition comprising an undissolved macromolecule, 401, is placed in a first chamber, 410, of a device of the present invention. The concentration of undissolved macromolecule in the first chamber is indicated by [DE]. Upon injection of a fluid medium into the first chamber the undissolved macromolecule, 401, undergoes irreversible dissolution in the fluid medium with a rate indicated by k₄₁₁, to form a free macromolecule, 402, wherein the macromolecule is a dimeric or higher-order complex present in the first chamber, 410, having a concentration [DE¹]. The free macromolecule can undergo reversible or irreversible dissociation to yield free macromolecules, 403 and 404, respectively, with a rate indicated by k₄₁₂, wherein the free macromolecules have a concentration in the first chamber of [D¹] and [E¹], respectively. Dissociation of the dimeric or higher-order complex macromolecule, 402, to provide the macromolecules, 403 and 404, can be reversible (e.g., k₄₁₂≈k₄₁₂′) or irreversible (e.g., k₄₁₂>>k₄₁₂′). A semi-permeable barrier, 430, is selected such that macromolecule 404 does not substantially cross the semi-permeable barrier to the second chamber, 420. However, the free macromolecule 403 can pass through the semi-permeable barrier, 430, with a rate indicated by k₄₁₃, to provide the free macromolecule, 405, in the second chamber at a concentration [D²]. In some embodiments, the free macromolecule, 405, can pass back across the semi-permeable barrier from the second to the first chamber with a rate of k₄₁₃′. The concentration of free macromolecule in the second chamber, [D²], is measured as a function of time to determine a release profile for the undissolved macromolecule, 401, from a pharmaceutical composition.

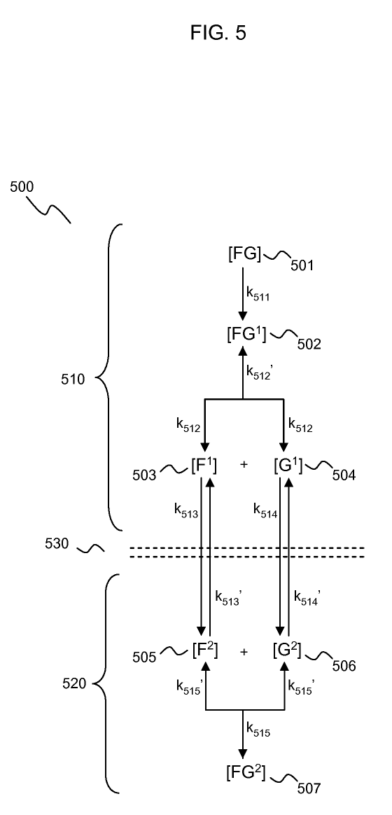

Referring to FIG. 5, a pharmaceutical composition comprising an undissolved macromolecule, 501, is placed in a first chamber, 510, of a device of the present invention. The concentration of undissolved macromolecule in the first chamber is indicated by [FG]. Upon injection of a fluid medium into the first chamber the undissolved macromolecule, 501, undergoes irreversible dissolution in the fluid medium with a rate indicated by k₅₁₁, to form a free macromolecule, 502, wherein the macromolecule is a dimeric or higher-order complex present in the first chamber, 510, having a concentration [FG¹]. The free macromolecule can undergo reversible or irreversible dissociation to yield free macromolecules, 503 and 504, respectively, with a rate indicated by k₅₁₂, wherein the free macromolecules have a concentration in the first chamber of [F¹] and [G¹], respectively. Dissociation of the dimeric or higher-order complex macromolecule, 502, to provide the macromolecules, 503 and 504, can be reversible (e.g., k₅₁₂≈k₅₁₂′) or irreversible (e.g., k₅₁₂>>k₅₁₂′). A semi-permeable barrier, 430, is selected such that both macromolecules, 503 and 504, can pass through the semi-permeable barrier to the second chamber, 420, with rates indicated by k₅₁₃ and k₅₁₄, respectively, to provide the free macromolecules, 505 and 506, respectively, in the second chamber at concentrations [F²] and [G²], respectively. In some embodiments, the free macromolecules, 505 and 506, can pass back across the semi-permeable barrier from the second to the first chamber with rates k₅₁₃′ and k₅₁₄′, respectively. Additionally, the free macromolecules, 505 and 506, can recombine in the second chamber with a rate, k₅₁₅ to provide a dimeric or higher-order complex macromolecule in the second chamber, 507. Complexation of the macromolecules, 505 and 506, in the second chamber, 520, to form a dimeric or higher-order complex macromolecule, 507, can be reversible (e.g., k₅₁₅≈k₅₁₅′) or irreversible (e.g., k₅₁₅>>k₅₁₅). Any one of the concentrations of the free macromolecules, 505 and 506, or the complex, 507, and combinations thereof, in the second chamber can be measured as a function of time to determine a release profile for a pharmaceutical composition comprising the undissolved macromolecule, 501, in the first chamber.

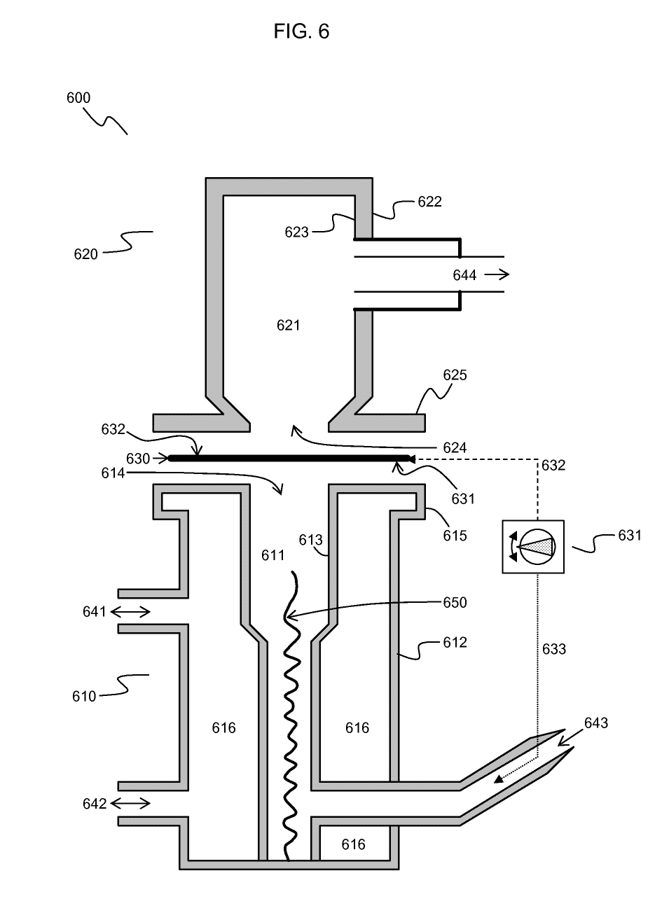

Referring to FIG. 6, a schematic cross-sectional representation of a device of the present invention is shown, the device, 600, including a first compartment, 610, and a second compartment, 620. The first compartment, 610, includes an exterior wall, 612, and an interior wall, 613, enclosing a first chamber, 611, having an opening, 614, with a flange, 615, surrounding the opening. The second compartment, 620, includes an exterior wall, 622, and an interior wall, 623, enclosing a second chamber, 621, having an opening, 624, with a flange, 625, surrounding the opening. The second compartment, 620, further comprises a sampling port, 644, suitable for removing an aliquot of a fluid medium present in the second chamber, 621.

The first and second compartments, 610 and 620, can be made from any inert and non-absorbent material such as, but not limited to, a glass, a plastic, a metal, and the like, a composite thereof, and combinations thereof. The first and second compartments can be made from the same or different materials. In some embodiments, the first and/or second compartments are constructed from a first material, and the surfaces of the first and/or second compartment(s) enclosing the first and second chamber(s), respectively, are coated with a second material. For example, a first compartment can be constructed from a metal having a glass coating on the surface enclosing the first chamber.

The first and second chambers, 621 and 622, respectively, can have any three-dimensional shape that includes an opening, such as, but not limited to, a cylindrical shape, a pyramidal shape, a cubic shape, a polygonal shape, a spherical shape, an ellipsoidal shape, and the like.

A semi-permeable barrier, 630, separates the first chamber, 611, from the second chamber, 621, such that a first surface of the semi-permeable barrier, 631, contacts the flange of the first compartment, 615, and a second surface of the semi-permeable barrier, 632, contacts the flange of the second compartment, 625. The flanges of the first and second compartments, 615 and 625, respectively, can be held together to form a fluid-impermeable seal (using, e.g., a clamp, adhesive, a pressure applied to one or both of the first and second compartments, a magnetic force, a static charge, and the like) such that the semi-permeable barrier, 630, is held in a fixed position between the first and second compartments.

In some embodiments, the first compartment, 610, further includes an jacket, 616, surrounding the first chamber, 611, the jacket having ports, 641 and 642, respectively, which can be used interchangeably as inlets and outlets. The jacket can be used for temperature control of the first compartment for example, by circulating a fluid (e.g., water, ethylene glycol, and the like) at a specified temperature (e.g., about 25° C., about 37° C., and the like) through the ports, 641 and 642, respectively. In some embodiments, the temperature of the first compartment is maintained at about 37° C. to simulate the temperature of the human body by circulating through the jacket, 616, a volume of water having a temperature of about 37° C.

The first compartment, 610, further includes an medium inlet, 643, permitting access to the first chamber, 611. The medium inlet, 643, can be used, for example, to introduce a fluid medium to the first chamber, 611, or to sample a volume of a fluid medium present in the first chamber, either directly or using an instrument.

In some embodiments, the device, 600, further includes a stirrer, 650, positioned in the first chamber, 611, suitable for mixing a fluid medium present in the first chamber.

In some embodiments, the device, 600, further comprises a control element, 631, suitable for applying an external force to the semi-permeable barrier, 630. The control element can be configured to directly apply the external force to the semi-permeable barrier, 632, or the external force can be applied to the semi-permeable through the fluid medium, 633.

### Methods

The present invention is also directed to a method of determining a release profile of an undissolved macromolecule from a pharmaceutical composition, the method comprising:
placing a pharmaceutical composition comprising an undissolved macromolecule in a device comprising a first compartment, wherein the first compartment is located adjacent to and in fluid communication with a second compartment, and wherein the first compartment is separated from the second compartment by a semi-permeable barrier adapted to prevent the undissolved macromolecule from passing from the first compartment to the second compartment;
filling the first compartment and the second compartment with a fluid, wherein the fluid interacts with the pharmaceutical composition to provide a free macromolecule in the first compartment, wherein a rate of transport of the free macromolecule from the first compartment to the second compartment is controlled by an interaction between the free macromolecule and the semi-permeable barrier;
measuring the concentration of the free macromolecule in the second compartment as a function of time; and
determining a release profile for the undissolved macromolecule, wherein the release profile is a function of at least the rate at which the free macromolecule is provided in the first compartment and the interaction between the free macromolecule and the semi-permeable barrier.

The present invention is also directed to a method for quantifying the stability and/or quality of a pharmaceutical composition comprising a macromolecule, the method comprising:
providing a first sample of a pharmaceutical composition comprising an undissolved macromolecule;
measuring a release profile for the first sample of the pharmaceutical composition comprising the macromolecule, the measuring comprising:
   (i) placing the pharmaceutical composition comprising an undissolved macromolecule in a device comprising a first compartment, wherein the first compartment is located adjacent to and in fluid communication with a second compartment, and wherein the first compartment is separated from the second compartment by a semi-permeable barrier adapted to prevent the undissolved macromolecule from passing from the first compartment to the second compartment;
   (ii) filling the first compartment and the second compartment with a fluid, wherein the fluid interacts with the pharmaceutical composition to provide a free macromolecule in the first compartment, and wherein a rate of transport of the free macromolecule from the first compartment to the second compartment is controlled by an interaction between the free macromolecule and the semi-permeable barrier;
   (iii) measuring the concentration of the free macromolecule in the second compartment as a function of time; and
   (iv) determining a release profile for the undissolved macromolecule, wherein the release profile is a function of at least the rate at which the free macromolecule is provided in the first compartment and the interaction between the free macromolecule and the semi-permeable barrier;
providing a second sample of the pharmaceutical composition comprising a macromolecule;
measuring a release profile for the second sample of the pharmaceutical composition comprising the macromolecule, the measuring comprising performing (i)-(iv) using the second sample of the pharmaceutical composition; and
comparing the release profile of the second sample with the release profile of the first sample of the pharmaceutical composition to quantify the stability and/or quality of the second sample of the pharmaceutical composition compared to the first sample of the pharmaceutical composition.

Referring to FIG. 6, a method for determining a release profile for a pharmaceutical composition comprising a macromolecule can be determined by placing a pharmaceutical composition in the first chamber, 611, and placing a semi-permeable barrier, 630, on the first flange, 615, surrounding the first opening, 614. As used herein, “placing” refers to physically locating the pharmaceutical composition within the first compartment. Referring to FIG. 6, placing can include inserting the pharmaceutical composition through the first opening, 614, or inserting the pharmaceutical composition through the medium inlet, 643.

In some embodiments, the method further comprises prior to the placing, measuring an amount of the pharmaceutical composition. Measuring an amount of the pharmaceutical composition can be performed by a method known to a person of ordinary skill in the art, or can be assumed based upon the addition of the entirety of a unit dosage to the first compartment.

Referring to FIG. 6, the second compartment, 620, is then placed over the first compartment, 610, by positioning the second flange, 625, to overlap with the surface of the first flange, thereby rigidly fixing the position of the semi-permeable barrier, 630, between the first and second flanges, 615 and 625, respectively. After a seal that is substantially impermeable to the fluid medium is formed between the first and second flanges, a fluid medium is introduced into the first chamber, 611 through the medium inlet, 643. The fluid medium fills the first chamber, 611. As used herein, “filling” refers to the first chamber being substantially void of free volume not occupied by the fluid.

The fluid medium substantially fills at least the first chamber, 611, and the semi-permeable barrier, 630. The second chamber, 621, is also filled with fluid, typically by passing fluid from the first chamber through the semi-permeable barrier, 630. In some embodiments, the sampling port, 644, can remain open while the first and second compartments are filled with a fluid to thus maintain an atmospheric pressure within the device. Typically, the filling of the first and second compartments with a fluid medium continues until the level of the fluid medium within the second compartment, 621, is at least above the level of the sampling port, 644. Subsequently the macromolecule present in the pharmaceutical composition will begin to dissolve in the fluid medium. Use of a mixing device, 650, can assist in dispersing the pharmaceutical composition within the first chamber, 611, and in some embodiments increase the dissolution rate of the macromolecule and/or pharmaceutical composition within the first chamber.

In some embodiments, the filling is performed prior to the placing such that a fluid medium is already present in the first and/or second chambers when the pharmaceutical composition is introduced into the first chamber.

It is not a requirement that excipients present in the pharmaceutical composition such as, but not limited to, diluents, fillers, solvents, glidants, surfactants, lubricants, emulsifiers, flavorants, anti-oxidants, and the like be completely dissolved in the fluid medium, or even that the excipients be substantially soluble in the fluid medium, so long as the macromolecule is capable of dissolving in the fluid medium.

The method further comprises measuring the concentration of the free macromolecule in the second compartment as a function of time. As used herein, “measuring” refers to an analytical determination of the amount of free macromolecule present in the second chamber. Measuring devices suitable for use with the present invention include, but are not limited to an electrical potentiostat, a spectrophotometer, a liquid chromatograph, a gas chromatography, a mass spectrometer, a gravimeter or gravitometer, a mass balance, and combinations thereof, and any other quantitative analytical means known to persons of ordinary skill in the analytical art.

In some embodiments, the concentration of the free macromolecule in the second chamber as a function of time can be determined by sampling the fluid in the second compartment followed by ex situ analysis. For example, the concentration of the macromolecule in the second chamber can be measured as a function of time utilizing, for example, the sampling port, 644, and a quantitative analysis technique such as, but not limited to, high performance liquid chromatography. In some embodiments, as samples are removed from the second chamber using the sampling port, 644, additional fluid medium can be supplied through the medium inlet, 643, such that the volume of fluid medium contained in the device is substantially constant. Introducing additional fluid medium can also facilitate the dissolution rate of the macromolecule from the pharmaceutical composition.

In some embodiments, the concentration of the free macromolecule in the second chamber can be performed by in situ analysis of the fluid in the second compartment, and combinations thereof. For example, the device can further include an optical window suitable for interfacing the device with a spectrophotometer and the like, or can be directly integrated with an analytical tool suitable for measuring a macromolecule concentration.

Not being bound by any particular theory, the concentration of the free macromolecule in the second chamber as a function of time depends on the rate at which the free macromolecule is provided within the first chamber, and the rate at which the free macromolecule passes through the semi-permeable barrier, 630. The rate of providing the free macromolecule can be largely controlled by temperature, the rate of stirring, one or more properties of the fluid medium, and the presence of adjuvants, moieties, and the like in the fluid medium that can enhance the dissolution rate of an undissolved macromolecule. The rate at which a free macromolecule passes through the semi-permeable barrier depends largely on an interaction between the free macromolecule and the semi-permeable barrier.

The interaction between the free macromolecule and the semi-permeable barrier can be enhanced or diminished by applying an external force to the semi-permeable barrier. Thus, in some embodiments the method further comprises applying an external force to the semi-permeable barrier, wherein the external force affects the interaction between the free macromolecule and the semi-permeable barrier. Thus, in some embodiments the device includes a control element, 631, suitable for applying and controlling an external force. An external force can be applied directly to the semi-permeable barrier, 632, or an external force can be applied to the semi-permeable barrier through the fluid medium, 633. In some embodiments, the control element can both apply an external force to the semi-permeable barrier and include an analytical instrument or measuring device for measuring the amplitude of the external force that is applied with a feedback control. For example, a control element can include a potentiostat, a galvanometer, a barometer, a pH meter, and the like, and any other analytical measurement device known to persons of ordinary skill in the art. In some embodiments, the control element can include a programmable circuit suitable for programming a routine or series predetermined levels at which the external force is applied to the device. In some embodiments, the control element comprises a personal computer or any other device having a graphical user interface.

Not being bound by any particular theory, the external force applied to the semi-permeable barrier can modify one or more properties of the semi-permeable barrier such as, but not limited to, the pore size and/or the degree of porosity of the barrier (e.g., by reversibly chemisorbing a species, moiety, and the like to the internal and/or external surfaces of the barrier), the volume of the semi-permeable barrier (e.g., by swelling or contracting the barrier), the presence and density of a functional group on the barrier (e.g., by inducing a reversible reaction at a functional group such as esterification, hydrolysis, amidification, and the like), the presence and amplitude of a static charge on the barrier (e.g., by inducing a local pH change, applying an electrical potential, and the like), and combinations thereof.

The determining comprises measuring the concentration of the macromolecule in the fluid medium present in the second compartment as a function of time.

The method further comprises determining a release profile for the undissolved macromolecule, wherein the release profile is a function of at least the rate at which the free macromolecule is provided in the first compartment and the interaction between the free macromolecule and the semi-permeable barrier.

As used herein, a “release profile of a pharmaceutical composition” or a “release profile of a macromolecule from a pharmaceutical composition” refers to an empirically determined function having the variable percent concentration versus time. The percent concentration determined as a function of time is a function of at least the dissolution rate of a macromolecule from a pharmaceutical composition of interest and the rate at which the macromolecule crosses a semi-permeable barrier. In some embodiments, a release profile of a macromolecule from a pharmaceutical composition is equivalent to the release rate of a macromolecule from a pharmaceutical composition.

In some embodiments, a release profile is a statistically reliable value. Thus, a series of percent concentration values can be determined at discrete time points and compared to calculate a mean, median, or average percent concentration value at a predetermined time point, wherein the mean, median, or average value having a standard deviation associated with it. Thus, in some embodiments the method further comprises: emptying the first and second compartments of the fluid; cleaning the first and second compartments and the semi-permeable barrier; and repeating the placing, the filling, the measuring, and the determining. And in some embodiments, the method further comprises determining a mean release profile having a standard deviation. A standard deviation can be calculated for each time point of a release profile.

In an embodiment, a release profile is determined and used to quantify the stability of a pharmaceutical composition by determining a first release profile for the pharmaceutical composition. In a preferred embodiment, the first release profile is determined by conducting multiple parallel release profile tests using the device or multiple devices of the present invention to provide a first release profile having a relative standard deviation. A period of time is permitted to pass after determining the first release profile (e.g., several hours, days, weeks, months, or years). A second release profile is determined on the same or a similar pharmaceutical composition that was produced at the same time as the first pharmaceutical composition. In a preferred embodiment, the second release profile is also determined by conducting multiple parallel tests to provide a second release profile having a relative standard deviation. The first and second release profiles are then compared, and if the second release profile overlaps with the first release profile, then this is confirmation of the stability of the pharmaceutical composition during the time interval. However, if the first and second release profiles do not overlap (e.g., the second release profile falls outside the standard deviation of the first release profile), then this is an indication that the pharmaceutical formulation is not stable over the period of time between the first and second release profiles were determined.

In some embodiments, the method further comprises: permitting a predetermined amount of time to elapse between the first measuring and the second measuring. Testing the release profile of a pharmaceutical composition with the device of the present invention has the advantage of ensuring quality control and stability of the product. For example, testing of a pharmaceutical composition can be performed at regular intervals to determine that the composition is stabile and retains its full activity during storage, packaging, shipment, and the like.

In some embodiments, the first and second samples are from the same manufacturing lot, in which the method of the present invention is suitable for testing the stability of the pharmaceutical composition over time. For example, in some embodiments a series of release profile measurements is made on a first sample of a pharmaceutical composition. The release profile comprises a series of percent concentration values at predetermined time points, wherein each percent concentration value has a mean, median or average value associated with it and a standard deviation associated with the value. After the first series of release profile measurements are made a predetermined amount of time is elapsed (e.g., 1 week, 1 month, 2 months, 3 months, 6 months, 1 year, etc.), and a second series of release profiles are determined using a second sample of the same pharmaceutical composition. The second sample can be produced at the same time, before or after the first sample. The second series of release profiles are then averaged to provide a second mean, median, or average value having a standard deviation. The first and second release profiles are then compared, and the degree of similarity between the release profiles determines whether the bioavailability or in vivo dissolution rate of the first and second samples will be similar or differ.

In some embodiments, a release profile comprises a graphical plot of percent concentration of a macromolecule present in a pharmaceutical composition (y-axis) versus time (x-axis), wherein the lower limit for any x-axis value is 0, the minimum possible y-axis value is 0%, and when the minimum y-axis value corresponds to x=0, and wherein the upper limit for any y-axis value is 100%, and the upper limit for any x-axis value is the maximum time duration over which a dissolution measurement is performed (e.g., 1 minutes, 2 minutes, 5 minutes, 10 minutes, 60 minutes, 100 minutes, 120 minutes, 300 minutes, 10 hours, 12 hours, 15 hours, 18 hours, 24 hours, 28 hours, etc.).

In some embodiments, a “release profile of a pharmaceutical composition” or a “release profile of a macromolecule from a pharmaceutical composition” can be described by a linear, first order polynomial, second order polynomial, exponential, power series, or other equation. In some embodiments, a “best fit” curve can be determined for a release profile, wherein the best fit has an R-squared value (“R²”) of about 0.95 or more, about 0.96 or more, about 0.97 or more, about 0.98 or more, about 0.99 or more, or about 0.995 or more.

In some embodiments, a release profile has statistical relevance in that a first release profile determined for a pharmaceutical composition at a first point in time can be compared with a second release profile determined for the same pharmaceutical composition at a second point in time, and differences between the first and second release profiles can be used for purposes of quality control such as, but not limited to, determining: product stability, bioequivalence, shelf-life, activity, potency, and the like.

Not being bound by any particular theory, determination of a release profile for a pharmaceutical composition from dissolution raw data (i.e., x=time, y=% concentration) permits facile comparison of release profile data from different experiments performed on the same or similar pharmaceutical compositions. Moreover, the release profiles can be statistically analyzed to compare data from different pharmaceutical formulations, or comparison of dissolution rate date from the same pharmaceutical formulation at different points in time.

In some embodiments, the method further comprises correlating the release profile of the macromolecule with a property (e.g., a physical property) of the pharmaceutical composition. For example, the release profile of a macromolecule from a pharmaceutical composition can be correlated with a property of the pharmaceutical composition such as, but not limited to: a solvent present in the pharmaceutical composition, the water content of the pharmaceutical composition, the oxidative stability of the pharmaceutical composition, the concentration of the macromolecule present in the pharmaceutical composition, the density, hardness, compaction, or friability of a pharmaceutical composition, and combinations thereof. In some embodiments, modification of one or more of these properties of the pharmaceutical composition can alter the release profile as determined by the method and device of the present invention.

In some embodiments, the method further comprises correlating the release profile of the macromolecule with an in vivo dissolution rate of the pharmaceutical composition.

Determination of a release profile using the device of the present invention can also be used to formulate a pharmaceutical composition having a predetermined in vivo dissolution rate and/or bioavailability. For example, matching of the release profile of a second pharmaceutical composition with a release profile of a first pharmaceutical composition can be used as an indication that the two pharmaceutical compositions are bioequivalent.

In some embodiments, the method further comprises correlating the release profile of the macromolecule with a bioavailability of the macromolecule. For example, in some embodiments, a release profile of a macromolecule from a pharmaceutical composition determined by the method of the present invention correlates with a Cₘₐₓ of the macromolecule or a metabolite thereof, a Tₘₐₓ of the macromolecule or a metabolite thereof, an AUC₍₀₋ₜ₎ of the macromolecule or a metabolite thereof, an AUC_{∞} of the macromolecule or a metabolite thereof, and combinations thereof, upon administration of the pharmaceutical composition comprising the macromolecule to a subject in need thereof.

As used herein, “Cₘₐₓ” refers to the maximum concentration of the macromolecule or an active metabolite thereof, observed after administration of a pharmaceutical composition comprising the macromolecule to a subject in need thereof.

As used herein, “Tₘₐₓ” refers to the time at which the maximum concentration of the macromolecule or an active metabolite thereof is achieved after administration of a pharmaceutical composition comprising the macromolecule to a subject in need thereof.

As used herein, “AUC₍₀₋ₜ₎” refers to the Area Under the Concentration time curve (i.e., plot of plasma concentration vs. time) after administration of a pharmaceutical composition comprising the macromolecule to a subject in need thereof. The area is conveniently determined by the “trapezoidal rule”: the data points are connected by straight line segments, perpendiculars are erected from the abscissa to each data point, and the sum of the areas of the triangles and trapezoids so constructed is computed.

As used herein, “AUC_{∞}” refers to the Area Under the Concentration time curve, wherein the last concentration is extrapolated to baseline based on the rate constant for elimination.

In some embodiments, the method further comprises: emptying the first and second compartments of the fluid; cleaning the first and second compartments; replacing the semi-permeable barrier with a second semi-permeable barrier, wherein the second semi-permeable barrier is different from the semi-permeable barrier; and repeating the placing, the filling, the measuring, and the determining.

The present invention is also directed to a method of determining a release profile of a crystalline insulin from a pharmaceutical composition, the method comprising:
placing the pharmaceutical composition in a device comprising a first compartment, wherein the first compartment is located adjacent to and in fluid communication with a second compartment, wherein the first compartment is separated from the second compartment by a semi-permeable barrier adapted to prevent the crystalline insulin from passing from the first compartment to the second compartment;

filling the first compartment and the second compartment with a fluid, wherein the fluid interacts with the pharmaceutical composition to provide a free insulin in the first compartment, wherein a rate of transport of the free insulin from the first compartment to the second compartment is controlled by an interaction between the free insulin and the semi-permeable barrier;
measuring the concentration of the free insulin in the second compartment as a function of time; and
determining a release profile for the crystalline insulin from the pharmaceutical composition, wherein the release profile is a function of at least rate at which the free insulin is provided in the first compartment and the interaction between the free insulin with the semi-permeable barrier.

In some embodiments, the method of determining a release profile of a crystalline insulin from a pharmaceutical composition is performed using a cellulose acetate membrane.

### EXAMPLES

### Example 1

Insulin is a peptide hormone composed of 51 amino acid residues, having a molecular weight of 5808 Daltons. Insulin is available as a rapid-acting formulation (i.e., action begins immediately and lasts about 3-4 hours), a short-acting formulation (i.e., action begins within 30 minutes and lasts about 5-8 hours), an intermediate-acting formulation (i.e., action begins within about 1-3 hours and lasts about 16-24 hours), and a long-acting formulation (action begins within about 4-6 hours and lasts 24-28 hours). Additional “mixed-action formulations” having a customized release profile can be achieved by mixing various amounts of the rapid-, short-, intermediate-, and long-acting formulations.

Insulin R is mixed-action insulin formulation that acts rapidly and has a duration of activity of about 4-12 hours. Insulin N (NPH insulin) is an isophane suspension (i.e., a crystalline suspension of human insulin in the presence of Zn²⁺ ions and protamine) that exhibits an extended release profile with a slower onset of action than insulin R. Mixed-action insulin formulations can also be constituted by mixing insulin R with an appropriate amount of insulin N to obtain a desired release profile. For example, “insulin 50/50” refers to a composition comprising 50% insulin N and 50% insulin R, while “insulin 70/30” refers to a composition comprising 70% insulin N and 30% insulin R. The concentration of insulin released from an arbitrary insulin formulation comprised of insulin R and insulin N as a function of time can be predicted using equation (1):

$$x ( t ) = R ( t ) \times ( \frac{x}{100} ) + ( 100 - N ) \qquad ( 1 )$$

wherein x(t) is the predicted percentage of insulin released at time t from an arbitrary pharmaceutical composition x, R(t) is the observed percentage of insulin released at time t from an insulin N formulation, and N is the percent concentration of insulin N present in formulation x (i.e., x=70 for insulin 70/30, x=50 for insulin 50/50 and x=0 for insulin R).

The release profile of various insulin formulations (insulin N, insulin 70/30 and insulin 50/50) were tested in a device similar to that depicted in FIG. 6, wherein the semi-permeable barrier, 630, was a cellulose acetate barrier having a porosity of 0.45 μm. Fluid samples were taken from the second compartment at predetermined time intervals and the concentration of insulin in the fluid medium was determined by high-performance liquid chromatography (“HPLC”). This concentration of insulin in the fluid medium present in the second compartment was compared with the total concentration of insulin present in the insulin formulation per the labeled claim, and the percentage of the labeled claim (“% LC”) was calculated according to equation (2):

$$\% L C = 100 \% - ( \frac{L C ( x , n ) - Rx ( t , n )}{L C ( x , n )} ) \times 100 \% \qquad ( 2 )$$

wherein LC(x,n) is the total amount (i.e., moles or mass) of a macromolecule present in pharmaceutical composition x divided by the total volume of the test fluid present in the testing apparatus, Rx(t,n) is the observed concentration of macromolecule (i.e., moles/liter or grams/liter) present in the test fluid sampled at time t, and wherein a % LC at time t for pharmaceutical composition x is the arithmetic mean of n samples. A standard deviation, a variance, and the like can also be calculated for % LC as a function of the distribution of the n data points at time t for pharmaceutical composition x. The insulin release profile data thus obtained is listed in Tables 1-3.

TABLE 1
Dissolution testing results for Insulin N.

| | Insulin N (% LC) | | | | |
|---|---|---|---|---|---|
| Time (min) | Cell A | Cell B | Mean | RSD | Normalized |
| 0 | 0 | 0 | 0 | 0 | 0 |
| 2 | 0 | 0 | 0 | n/a | 0 |
| 12 | 13 | 12 | 12 | 4.6 | 12 |
| 22 | 42 | 40 | 41 | 3.3 | 38 |
| 32 | 93 | 90 | 92 | 2.1 | 85 |
| 42 | 109 | 108 | 108 | 0.9 | 101 |
| 52 | 107 | 107 | 107 | 0.3 | 100 |

TABLE 2
Dissolution testing results for Insulin 70/30.

| | Insulin 70/30 (% LC) | | | | |
|---|---|---|---|---|---|
| Time (min) | Cell A | Cell B | Mean | RSD | Normalized |
| 0 | 0 | 0 | 0 | 0 | 0 |
| 2 | 30 | 31 | 31 | 3.6 | 30 |
| 12 | 43 | 44 | 43 | 2.6 | 43 |
| 22 | 65 | 68 | 67 | 3.3 | 66 |
| 32 | 97 | 101 | 99 | 2.4 | 97 |
| 42 | 100 | 111 | 106 | 7.4 | 104 |
| 52 | 99 | 104 | 102 | 3.3 | 100 |

TABLE 3
Dissolution testing results for Insulin 50/50.

| | Insulin 50/50 (% LC) | | | | |
|---|---|---|---|---|---|
| Time (min) | Cell A | Cell B | Mean | RSD | Normalized |
| 0 | 0 | 0 | 0 | 0 | 0 |
| 2 | 51 | 53 | 52 | 2.6 | 51 |
| 12 | 58 | 60 | 59 | 2.0 | 58 |
| 22 | 75 | 76 | 75 | 0.7 | 74 |
| 32 | 100 | 102 | 101 | 1.4 | 99 |
| 42 | 106 | 110 | 108 | 2.1 | 106 |
| 52 | 99 | 105 | 102 | 3.8 | 100 |

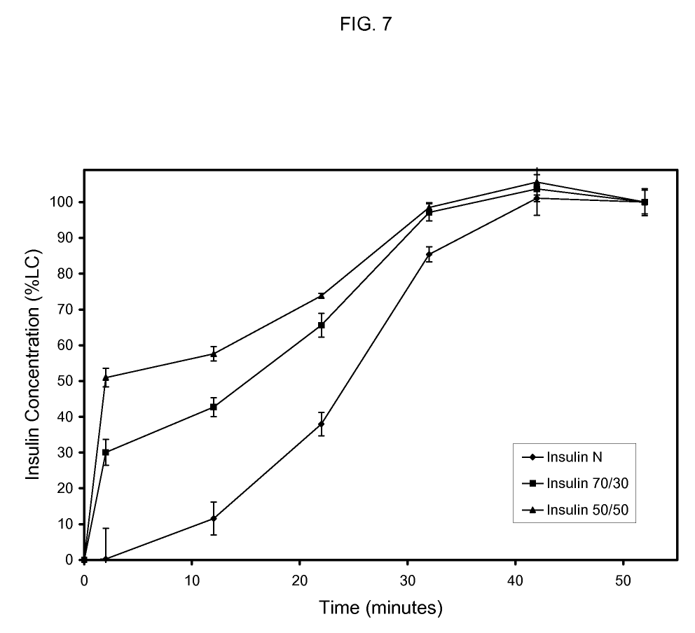

A drug release profile for each formulation was determined by plotting % LC (y-axis) versus time (x-axis). The drug release profile for each of the formulations is shown in FIG. 7. Referring to FIG. 7, a mean drug release profile for each insulin formulation: insulin N ( [symbol] ), insulin 70/30 ( [symbol] ), and insulin 50/50 ( [symbol] ) can be readily distinguished using a device and method of the present invention. Also shown in FIG. 7 is the estimated standard deviation for each release profile. The data show that the insulin formulations can be readily distinguished from one another by the device and method of the present invention. Specifically, there is no overlap of any of the drug release profiles for the various insulin formulations until after 40 minutes of testing.

### Example 2

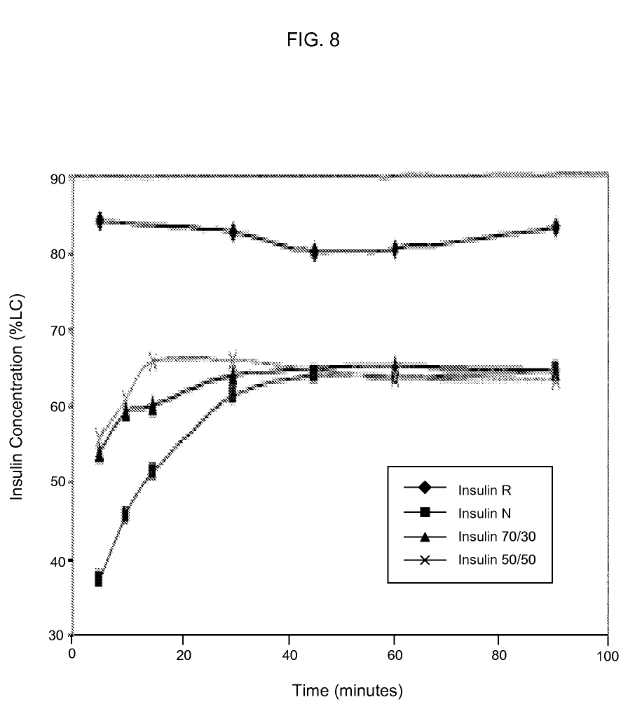

The dissolution rate of insulin from pharmaceutical compositions (insulin R, insulin N, insulin 70/30 and insulin 50/50) were measured using a paddle method, generally described in The United States Pharmacopeia 29 2673-2677 (The United States Pharmacopeia Convention, Inc., Rockville, Md.) (2006), which is incorporated herein by reference. The insulin containing pharmaceutical compositions were tested separately by placing 11.3 mg of the compositions in a cylindrical vessel which consists of a paddle formed from a blade and a shaft is used as the stirring element. Insulin suspension is allowed to sink to the bottom of the vessel and sealed before rotation of the blade is started. A fluid medium (900 mL of solution contains 5.7 mM sodium chloride, 1.2 mM potassium chloride and 29.4 mM phosphate) was added to the sealed vessel and the stirring paddle was rotated at 50 rpm. Insulin dissolved by the fluid medium was taken at predetermined time intervals to determine the insulin concentration released by the pharmaceutical composition. The insulin release rate (as % LC) was calculated as described above, the results of which are displayed graphically in FIG. 8.

Referring to FIG. 8, the drug release profile for insulin R ( [symbol] ) is nearly constant (i.e., complete dissolution occurs rapidly), while the drug release profiles of insulin N ( [symbol] ), insulin 70/30 ( [symbol] ) and insulin 50/50 ( [symbol] ) cannot be easily distinguished from one another. This is likely due to errors arising from using a device to quantify a release profile wherein a low concentration of a macromolecule in the fluid medium must be detected. Additionally, the high volume of fluid medium required for the paddle method device results in rapid dissolution of the macromolecule that leads to a large experimental error. Furthermore, because the vessel is sealed while conducting the trial, it can be difficult to introduce additional fluid medium to replenish that which is removed during sampling. Failure to maintain a constant volume of fluid can result in incomplete dissolution of the pharmaceutical composition, which is also observed in FIG. 8 for the insulin N, insulin 70/30 and insulin 50/50 formulations.

### Example 3

The dissolution rate of insulin from pharmaceutical compositions (insulin R and insulin N) were measured using a flow-through dissolution testing device, generally described in The United States Pharmacopeia 29:2678-2679 (The United States Pharmacopeia Convention, Inc., Rockville, Md.) (2006), which is incorporated herein by reference. The insulin containing pharmaceutical compositions were tested separately by placing 37.6 mg of the composition in a cylindrical flow-through cell having a first end that included a filter attached thereto and a second end that was open, but blocked by a moveable glass bead. The total internal volume of the flow-through cell was approximately 5 mL. A fluid medium, phosphate-buffered saline (PBS) solution (17.2 mM sodium chloride, 3.6 mM potassium chloride and 88.2 mM phosphate), pH 7.4, was flowed through the cell at a flow rate of 4 mL/min using a pump.

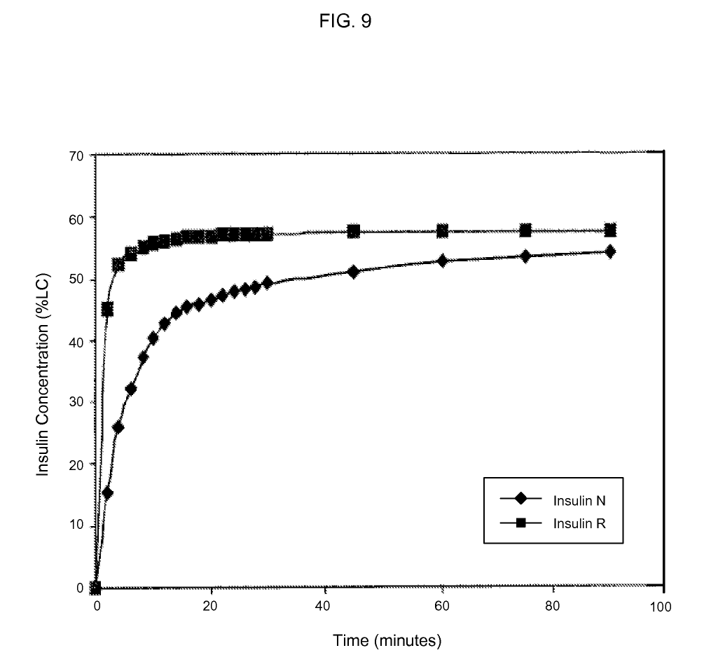

The pharmaceutical composition dissolved as the fluid medium passed through the flow-through cell. The release profile of insulin from the pharmaceutical compositions was measured as a function of time by measuring the insulin concentration in fluid contained within the vessel. The concentration of insulin was determined as a percentage of the labeled claim (% LC) versus time, the results of which are plotted in FIG. 9.

Referring to FIG. 9, the release profile curves for the insulin R ( [symbol] ) and insulin N ( [symbol] ) pharmaceutical compositions fail to reach a drug release of 100% LC. This likely results from particles of the pharmaceutical composition adhering to the filter of the flow-through dissolution testing device. Additional errors in determination of a release profile for a macromolecular composition arise from a high fluid volume and high fluid flow rate required for a flow-through device, which can result in greater sampling error and rapid dissolution rates, respectively. These factors can be partially accounted for by using a higher overall sample concentration, conducting dissolution concentration measurements at smaller time intervals, and/or increasing the number of dissolution trials.

However, the high cost of many macromolecular pharmaceutical compositions weighs against these options.

### Example 4

The release profile of a crystalline suspension of insulin (insulin N) was determined using a device of the present invention as a function of buffer concentration using a PBS solution. A device similar to that depicted in FIG. 6 was loaded with a pharmaceutical composition containing insulin (insulin N, 7.5 mg), the device was sealed, and the device was then filled with a PBS solution (sodium chloride, potassium chloride, dibasic sodium phosphate and potassium phosphate in water, pH=7.4). The total internal volume of the device was approximately 7 mL. The release profile of the insulin N composition was determined at PBS solution concentrations of 100% ( ), 75% ( [symbol] ), 50% ( [symbol] ) and 25% ( [symbol] ), wherein the 100% buffered aqueous fluid contained 15 mM sodium chloride, 0.34 mM potassium chloride and 176 mM phosphate. The lower buffer concentrations were prepared by diluting the 100% PBS solution with appropriate amounts of water. The fluid in the first compartment was stirred during the dissolution tests.

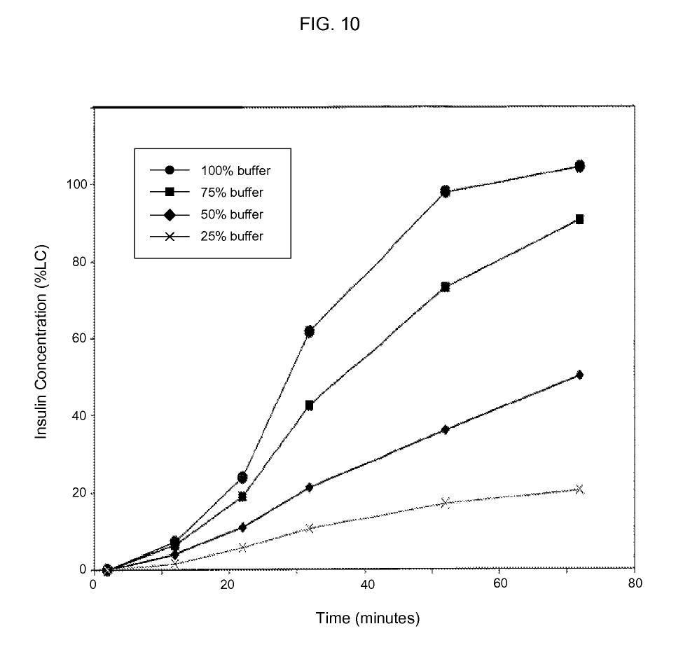

The insulin concentration in the second compartment of the device was measured at predetermined intervals, and the percentage of insulin released from the pharmaceutical composition was determined as a percentage of the labeled claim (% LC) versus time. The release profile for the pharmaceutical composition at each buffer concentration is plotted in FIG. 10. As shown in FIG. 10, an increase in buffer concentration resulted in a more rapid release profile for the insulin.

Not being bound by any particular theory, the release profile of an insulin pharmaceutical composition exhibits a more rapid release as the buffer concentration is increased because zinc ions present in the crystalline insulin are able to complex with the phosphate ions in the buffer to assist in the dissolution of the insulin. Accordingly, the rate of insulin dissolution is dependent on the phosphate concentration in the fluid medium. This effect should be observed similarly for any ions in solution capable of forming a complex with Zn²⁺ ions in a manner analogous to phosphate.

### Example 5

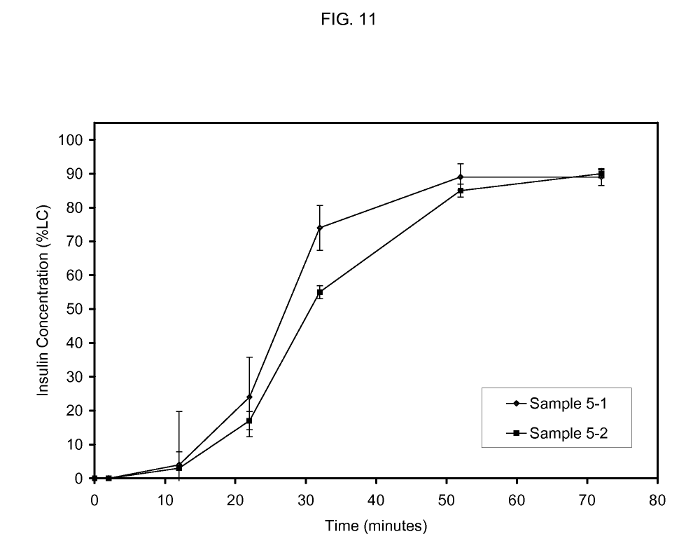

The stability of a pharmaceutical composition comprising insulin N was determined over a 3-month time period. Two identical insulin N pharmaceutical compositions from the same manufacturing lot (“Sample 5-1” and “Sample 5-2”) were prepared and placed in glass vials. The release profile of Sample 5-1 was determined using the procedure described in Example 1, the results of which are shown graphically in FIG. 11 ( [symbol] ). The vial containing Sample 5-2 was stored for 3 months at 25° C. in a box, at which time the release profile for Sample 5-2 was determined, the results of which are shown graphically in FIG. 11 ( [symbol] ). Also shown in FIG. 11 are the standard deviation data for each of the release profiles. Referring to FIG. 11, the release profile for Sample 5-2 falls within one standard deviation of the release profile for Sample 5-1. However, the lack of overlap between the release profiles at 32 minutes suggests there was a physical and/or chemical change in insulin N Sample 5-2 during storage. Thus, the method and device of the present invention provides a sensitive means for investigating the stability compositions comprising macromolecules.

### CONCLUSION

These examples illustrate possible embodiments of the present invention. While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

It is to be appreciated that the Detailed Description section, and not the Summary and Abstract sections, is intended to be used to interpret the claims. The Summary and Abstract sections can set forth one or more, but not all exemplary embodiments of the present invention as contemplated by the inventor(s), and thus, are not intended to limit the present invention and the appended claims in any way.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A device comprising:
a first compartment configured to receive a pharmaceutical composition comprising an undissolved macromolecule and a fluid suitable for dissolving the macromolecule to provide a free macromolecule;
a second chamber adjacent to the first chamber, wherein the first chamber and the second chamber are in fluid communication;
a semi-permeable barrier separating the first and the second chambers, wherein a rate of transport of the free macromolecule from the first chamber to the second chamber is determined by an interaction between the free macromolecule and the semi-permeable barrier; and wherein the semi-permeable barrier is adapted to prevent the undissolved macromolecule from passing from the first chamber to the second chamber; and
a sampling port configured to sample the contents of the second chamber.

2. The device of claim 1, further comprising a control element configured to apply an external force to the semi-permeable barrier.

3. The device of claim 2, wherein the control element is configured to apply an external force to the semi-permeable barrier chosen from: an electrical potential, a direct electric current, an alternating electric current, a magnetic field, a pressure, a thermal energy, a radiation, a chemical reagent, and combinations thereof.

4. The device of claim 2, wherein the external force is applied to the semi-permeable barrier via the fluid.

5. The device of claim 1, wherein the semi-permeable barrier comprises a material chosen from: a polymer, a glass, a metal, a natural tissue, a synthetic tissue, a biological sample, a composite thereof, and combinations thereof.

6. The device of claim 1, further comprising a temperature control element surrounding at least the first chamber.

7. The device of claim 1, further comprising a stirrer configured to agitate a fluid within the first chamber.

8. The device of claim 1, wherein the first chamber is located below the second chamber.

9. The device of claim 1, further comprising a means for measuring an in situ concentration of a macromolecule present in the second chamber.

10. The device of claim 1, wherein the volume of the first chamber is about 50 mL or less and wherein the volume of the second chamber is about 50 mL or less.

11. A method of determining a release profile of an undissolved macromolecule from a pharmaceutical composition, the method comprising:
placing a pharmaceutical composition comprising an undissolved macromolecule in a device comprising a first chamber, wherein the first chamber is located adjacent to and in fluid communication with a second chamber, and wherein the first chamber is separated from the second chamber by a semi-permeable barrier adapted to prevent the undissolved macromolecule from passing from the first chamber to the second chamber;
filling the first chamber and the second chamber with a fluid, wherein the fluid interacts with the pharmaceutical composition to provide a free macromolecule in the first chamber, wherein a rate of transport of the free macromolecule from the first chamber to the second chamber is controlled by an interaction between the free macromolecule and the semi-permeable barrier;
measuring the concentration of the free macromolecule in the second chamber as a function of time; and
determining a release profile for the undissolved macromolecule, wherein the release profile is a function of at least the rate at which the free macromolecule is provided in the first chamber and the interaction between the free macromolecule and the semi-permeable barrier.

12. The method of claim 11, further comprising applying an external force to the semi-permeable barrier, wherein the external force affects the interaction between the free macromolecule and the semi-permeable barrier.

13. The method of claim 11, further comprising controlling the temperature of at least one of: the fluid, the first chamber, the second chamber, and the semi-permeable barrier.

14. The method of claim 11, further comprising:
emptying the first and second chambers of the fluid;
cleaning the first and second chambers and the semi-permeable barrier; and
repeating the placing, the filling, the measuring, and the determining.

15. The method of claim 11, further comprising:
emptying the first and second chambers of the fluid;
cleaning the first and second chambers;
replacing the semi-permeable barrier with a second semi-permeable barrier, wherein the second semi-permeable barrier is different from the semi-permeable barrier; and
repeating the placing, the filling, the measuring, and the determining.

16. The method of claim 11, further comprising correlating the release profile of the macromolecule with a physical property of the pharmaceutical composition.

17. The method of claim 11, further comprising correlating the release profile of the macromolecule with an in vivo dissolution rate of the pharmaceutical composition.

18. The method of claim 11, further comprising correlating the release profile of the macromolecule with a bioavailability of the macromolecule.

19. A method of determining a release profile of a crystalline insulin from a pharmaceutical composition, the method comprising:
placing the pharmaceutical composition in a device comprising a first chamber, wherein the first chamber is located adjacent to and in fluid communication with a second chamber, wherein the first chamber is separated from the second chamber by a semi-permeable barrier adapted to prevent the crystalline insulin from passing from the first chamber to the second chamber;
filling the first chamber and the second chamber with a fluid, wherein the fluid interacts with the pharmaceutical composition to provide a free insulin in the first chamber, wherein a rate of transport of the free insulin from the first chamber to the second chamber is controlled by an interaction between the free insulin and the semi-permeable barrier;
measuring the concentration of the free insulin in the second chamber as a function of time; and
determining a release profile for the crystalline insulin from the pharmaceutical composition, wherein the release profile is a function of at least rate at which the free insulin is provided in the first chamber and the interaction between the free insulin with the semi-permeable barrier.

20. A method for quantifying stability or quality of a pharmaceutical composition comprising a macromolecule, the method comprising:
providing a first sample of a pharmaceutical composition comprising an undissolved macromolecule;
measuring a release profile for the first sample of the pharmaceutical composition comprising the macromolecule, the measuring comprising:
(i) placing the pharmaceutical composition comprising an undissolved macromolecule in a device comprising a first chamber, wherein the first chamber is located adjacent to and in fluid communication with a second chamber, and wherein the first chamber is separated from the second chamber by a semi-permeable barrier adapted to prevent the undissolved macromolecule from passing from the first chamber to the second chamber;
(ii) filling the first chamber and the second chamber with a fluid, wherein the fluid interacts with the pharmaceutical composition to provide a free macromolecule in the first chamber, and wherein a rate of transport of the free macromolecule from the first chamber to the second chamber is controlled by an interaction between the free macromolecule and the semi-permeable barrier;
(iii) measuring the concentration of the free macromolecule in the second chamber as a function of time; and
(iv) determining a release profile for the undissolved macromolecule, wherein the release profile is a function of at least the rate at which the free macromolecule is provided in the first chamber and the interaction between the free macromolecule and the semi-permeable barrier;
providing a second sample of the pharmaceutical composition comprising a macromolecule;
measuring a release profile for the second sample of the pharmaceutical composition comprising the macromolecule, the measuring comprising performing (i)-(iv) using the second sample of the pharmaceutical composition; and
comparing the release profile of the second sample with the release profile of the first sample of the pharmaceutical composition to quantify the stability and/or quality of the second sample of the pharmaceutical composition compared to the first sample of the pharmaceutical composition.

21. The method of claim 20, further comprising waiting a predetermined time interval between the measuring a release profile for the first sample of the pharmaceutical composition and the measuring a release profile for the second sample of the pharmaceutical composition.

22. The method of claim 20, wherein the first sample of the pharmaceutical composition and the second sample of the pharmaceutical composition are from the same manufacturing lot.
